(19) United States
(12) Patent Application Publication
(10) Pub. No.: US 2009/0142303 A1
(43) Pub. Date: Jun. 4, 2009

(54) METHODS AND COMPOSITIONS FOR DRIED CELLULAR FORMS

(76) Inventors: David Edwards, Boston, MA (US); Yun-Ling Wong, Cambridge, MA (US); Brian Pulliam, Cambridge, MA (US); Kevin Kit Parker, Cambridge, MA (US); Sean Sheehy, Arlington, MA (US)

Correspondence Address: FISH & RICHARDSON PC, P.O. BOX 1022, MINNEAPOLIS, MN, 55440-1022, US

(21) Appl. No.: 12/063,485

(22) PCT Filed: Aug. 11, 2006
(86) PCT No.: PCT/US06/31580

§ 371 Date: Nov. 12, 2008

Related U.S. Application Data
(60) Provisional application No. 60/707,425, filed on Aug. 11, 2005.
(60) Provisional application No. 60/788,133, filed on Mar. 31, 2006.

(51) Int. Cl. A61K 35/74; A61K 35/12; A61K 35/14; A61K 35/76; A61P 43/00

(52) U.S. Cl. 424/93.4; 424/93.7; 424/93.72; 424/93.6

(57) ABSTRACT

Methods and compositions of spray drying cellular material are provided that allow preservation of the cellular material. In one aspect, the cellular material is spray dried with a quantity of excipient. In another aspect, the cellular material is spray dried using a cryoprotectant.

### CROSS-REFERENCE TO RELATED APPLICATIONS

This application claims priority to U.S. Application Ser. No. 60/707,425, filed on Aug. 11, 2005, and U.S. Application Ser. No. 60/788,133, filed Mar. 31, 2006. The entire contents of both prior applications are incorporated herein by reference.

### BACKGROUND

Dry forms of viral particles, cellular organisms, and other membrane bound materials can be of great utility in the pharmaceutical and general healthcare industries. Dry cellular forms (DCF) exhibit the utility of long-term storage, ease of processing, and delivery for food, agriculture, and human health applications. Examples of DCF include dry yeast for food applications, cryopreserved cells (for instance blood cells), and whole cells for gene delivery (Trsic-Milanovic et al., J. Serb. Chem. Soc., 66:435-42, 2001; Diniz-Mendes et al., Biotechnol. Bioeng., 65:572-8, 1999; and Seville et al., J. Gene Med., 4:428-37, 2002).

DCF are typically prepared by two methods: i) lyophilization or freeze drying, which involves bulk drying of aqueous suspensions of the cellular form or ii) cryopreservation, which involves the infusion of high levels of cryoprotectant into the aqueous cellular suspensions and lowering the temperature of the suspension to below 0° C. at a prescribed rate that minimizes cell death. One disadvantage of lyophilization (or freeze drying) and cryopreservation is the difficulty in preparing DCF in large volumes at a low cost while preserving the majority of the cellular material (Kirsop and Snell, eds., 1984, Maintenance of Microorganisms: A Manual of Laboratory Methods, London, Academic Press). Both techniques are limited by mass transfer across the lipid bilayer membrane and related osmotic stresses.

Lyophilization is used in the commercial preparation of Bacillus Calmette-Guerin (BCG) vaccine. BCG is given via injection to millions of newborn infants annually to protect against tuberculosis (TB), a disease caused by a bacterium called the tubercle bacillus or Mycobacterium tuberculosis (Roche et al., Trends Microbiol, 3:397-401, 1995). Presently, TB is the sixth largest cause of death and the global epidemic is growing at an estimated annual rate of 3%. The emergence of AIDS and its liaison with TB have brought an increased urgency for a new vaccine, since BCG is only moderately effective over the time period of a person's vulnerability to TB infection, typically the first 30 years of a person's life (Fine, Lancet, 346:1339-1345, 1995). One potential reason for the lack of efficacy of BCG is low viability of BCG in the manufactured DCF.

### SUMMARY

The invention is based, in part, on the discovery of new methods and compositions of spray dried cellular material that exhibit significant product yield, high organism activity (e.g., viability), and good powder processing properties. The dry cellular forms, e.g., produced by the compositions and methods described herein, have a low water content and can be suitable for administration to a subject by inhalation. The dry cellular forms retain activity for a period of time when stored at temperatures above freezing, allowing for ease of storage (e.g., long-term storage) and delivery. These properties allow the methods and compositions described herein to be useful for vaccine preparations, e.g., to be administered by injection, oral administration, or inhalation.

In one aspect, the invention includes dry powders with less than about 10% (e.g., less than about 8%, 5%, 4%, 3%, 2%, or 1%) water, e.g., free water, a cellular material, and at least 25% (e.g., at least 30%, 40%, 50%, 60%, 70%, 80%, 90%, 92%, 94%, 96%, 98%, 99%, or greater) of an excipient by dry weight. In some embodiments, the powders are produced without freezing. In some embodiments, the powders are produced by spray drying. In some embodiments, the cellular material includes bacteria (e.g., bacteria of the genus Mycobacterium, e.g., M. tuberculosis, M. smegmatis, or Bacillus Calmette-Guerin), viruses, eukaryotic microbes, mammalian cells (e.g., red blood cells, stem cells, granulocytes, fibroblasts, or platelets), membrane-bound organelles, liposomes, membrane-based bioreactors, or membrane-based drug delivery systems. In some embodiments, the ratio of mass of excipient to number of units of cellular material is at least 0.25 pg of excipient per unit of cellular material (e.g., at least 0.25, 0.5, 1, 2, 5, 10, 20, 50, 100, 200, 500, 1000, 2000, 5000, 10,000, or 20,000 pg of excipient per unit of cellular material). In some embodiments, the ratio of mass of excipient to mass of cellular material is at least 0.1 (e.g., at least 0.25, 0.5, 1, 2, 5, 10, 15, 20, 25, 30, 40, 50, 100, 200, 500, 1000, or 2000). In some embodiments when the powder includes live cells (e.g., bacteria), greater than 0.5% (e.g., 1%, 2%, 4%, 5%, 6%, 8%, 10%, 12%, 15%, 18%, 20%, 25%, or greater) of the cells are viable. In some embodiments, the live cells in the powder retain greater than 1/1000 (e.g., greater than 1/500, 1/200, 1/100, 1/50, 1/20, or 1/10) of their initial viability after storage at greater than 0° C. (e.g., greater than 4° C., 10° C., 20° C., 25° C., 30° C., 40° C., or 50° C.) for a period of greater than 10 days (e.g., 20, 30, 40, 50, 60, 70, 80, 90, 100, 110, or 120 days). In some embodiments, the excipient(s) include leucine, mannitol, trehalose, dextran, lactose, sucrose, sorbitol, albumin, glycerol, ethanol, or mixtures thereof. In some embodiments, the powders do not include cryoprotectant, e.g., added cryoprotectant or a significant amount of cryoprotectant (e.g., a cryoprotectant that is not the excipient). In some embodiments, the powders do not include salt, e.g., added salt or a significant amount of salt. The dry powders can be formulated as pharmaceutical compositions, e.g., for administration by inhalation.

In another aspect, the invention includes methods of producing dry powders that include cellular materials by providing an aqueous solution including at least 0.01 mg/ml (e.g., at least 0.1, 1, 2, 5, 10, 20, 50, 100, or 200 mg/ml) of excipient(s) and at least 10⁵ units/ml (e.g., at least 10⁶, 10⁷, 10⁸, 10⁹ or 10¹⁰ units/ml) of a cellular material, and spray-drying the solution under conditions to produce a dry powder that includes the cellular material with less than about 10% (e.g., less than about 8%, 5%, 4%, 3%, 2%, or 1%) water, e.g., free water, by weight. In some embodiments, the ratio of mass of excipient to number of units of cellular material is at least 0.25 picograms of excipient per unit of cellular material (e.g., at least 0.25, 0.5, 1, 2, 5, 10, 20, 50, 100, 200, 500, 1000, 2000, 5000, 10,000, or 20,000 pg of excipient per unit of cellular material). In some embodiments, the ratio of mass of excipient to mass of cellular material is at least 0.1 (e.g., at least 0.25, 0.5, 1, 2, 5, 10, 15, 20, 25, 30, 40, 50, 100, 200, 500, 1000, or 2000). In some embodiments wherein the cellular material includes bacteria (e.g., Gram-positive bacteria), the solution does not contain added salt or cryoprotectant. In some embodiments wherein the cellular material includes eukaryotic cells (e.g., mammalian cells), the solution can include salts or other solutes sufficient to minimize osmotic pressure.

In some embodiments, the solution includes least 10% (e.g., at least 25%, 30%, 40%, 50%, 60%, 70%, 80%, 90%, 92%, 94%, 96%, 98%, 99%, or greater) excipient by dry weight. In some embodiments, the solution includes less than 10¹⁰ units/ml (e.g., less than 10⁹, 10⁸, 10⁷, or 10⁶ units/ml) of a cellular material. In some embodiments, the cellular material includes bacteria (e.g., bacteria of the genus Mycobacterium, e.g., M. tuberculosis, M. smegmatis, or Bacillus Calmette-Guerin), viruses, eukaryotic microbes, mammalian cells (e.g., red blood cells, stem cells, granulocytes, fibroblasts, or platelets), membrane-bound organelles, liposomes, membrane-based bioreactors, or membrane-based drug delivery systems. In some embodiments, the excipient(s) include leucine, mannitol, trehalose, dextran, lactose, sucrose, sorbitol, albumin, glycerol, ethanol, or mixtures thereof. In some embodiments, the aqueous solution does not contain a cryoprotectant, e.g., a cryoprotectant that is not the excipient. In some embodiments, the methods further include formulating the dry powder in a pharmaceutical composition, e.g., for administration by inhalation. The invention also includes dry powders that include a cellular material that are produced by the new methods.

In another aspect, the invention includes methods of spray-drying a cellular material to minimize damage to the material by reducing osmotic stress. Osmotic stress can be reduced by obtaining an initial value for the radius of a unit of the cellular material (also referred to herein as a cell) to be spray dried (R^{c}(0)), selecting values for each of (i) difference in inlet and outlet gas temperatures of a spray dryer (ΔT), (ii) average droplet size (R^{d}), (iii) latent heat of vaporization of a solvent (λ), (iv) hydraulic permeability of a membrane of the cellular material to a cryoprotectant (Lₚ), (v) moles of extracellular solute (x^{e}ₛ), (vi) moles of intracellular solute (xⁱₛ), (vii) moles of extracellular cryoprotectant (x^{e}_{cp}), (viii) initial intracellular concentration of cryoprotectant (Cⁱ_{cp}(0)), and (ix) number of cells (n_{cells}), evaluating equation 36 using the selected values

$$- \frac{1}{{L}_{p} {R}_{gas} T} \frac{d {R}^{c} ( t )}{d t} = \frac{{x}_{s}^{e}}{4 / 3 π [ {( kt + {R}_{o}^{{d}^{2}} )}^{3 / 2} - {{n}_{cells} ( {R}^{c} ( t ) )}^{3} ]} - \frac{{x}_{s}^{i}}{4 / 3 π {{R}^{c} ( t )}^{3} - {V}_{excluded}} + σ [ \frac{{x}_{cp}^{e}}{4 / 3 π [ {( kt + {R}_{o}^{{d}^{2}} )}^{3 / 2} - {{n}_{cells} ( {R}^{c} ( t ) )}^{3} ]} - {C}_{cp}^{i} ( 0 ) ] 2 {\sum }_{n = 1}^{∞} \frac{{sin}^{2} ( {λ}_{n} ) - {λ}_{n} sin ( {λ}_{n} ) cos ( {λ}_{n} )}{{λ}_{n}^{2} - {λ}_{n} sin ( {λ}_{n} ) cos ( {λ}_{n} )} {e}^{- {λ}_{n}^{2} \overline{{D}_{cp}^{*} t} / {{R}^{c} ( t )}^{2}} \qquad ( 36 )$$

and, if R^{c}(t) is maintained within a minimum and maximum limit over a predicted drying time, spray drying the cellular material using the conditions of the selected values to minimize damage to the material. In some embodiments, the methods also include determining a predicted drying time. The minimum and maximum limit can be selected to minimize damage to the material. For example, the minimum limit can be at least about 60% (e.g., at least 70%, 80%, 90%, 95%, 98%, or 99%) of the initial radius.

For example, the maximum limit can be at most 160% (e.g., at most 140%, 125%, 110%, 105%, 102%, or 101%) of the initial radius. In some embodiments, the cellular material includes bacteria (e.g., bacteria of the genus Mycobacteriun, e.g., M. tuberculosis, M. smegmatis, or Bacillus Calmette-Guerin), viruses, eukaryotic microbes, mammalian cells (e.g., red blood cells, stem cells, granulocytes, fibroblasts, or platelets), membrane-bound organelles, liposomes, membrane-based bioreactors, or membrane-based drug delivery systems. In some embodiments, the cryoprotectant is added to the cellular material (e.g., inside or outside the cellular material) immediately prior to spray drying. In some embodiments, the methods further include formulating the dry powder in a pharmaceutical composition, e.g., for administration by inhalation. The invention also includes dry powders that include a cellular material that are produced by the new methods.

In yet another aspect, the invention includes methods of producing a dry powder including less than about 10% (e.g., less than about 8%, 5%, 4%, 3%, 2%, or 1%) water, e.g., free water, and bacteria of the genus Mycobacterium by providing an aqueous solution including at least 0.01 mg/ml (e.g., at least 0.1, 1, 2, 5, 10, 20, 50, 100, or 200 mg/ml) of excipient(s) and at least 105 colony forming units/ml (e.g., at least 10⁶, 10⁷, 10⁸, 10⁹, or 10¹⁰ colony forming units/ml) of bacteria of the genus Mycobacterium, and spray-drying the solution under conditions to produce a dry powder including less than about 10% (e.g., less than about 8%, 5%, 4%, 3%, 2%, or 1%) water, e.g., free water, and bacteria of the genus Mycobacterium. In some embodiments, the solution includes at least 0.25 pg of excipient per colony forming unit (e.g., at least 0.5, 1, 2, 5, 10, 15, 20, 25, 35, or 50 pg of excipient per colony forming unit) of bacteria of the genus Mycobacterium. In some embodiments, the aqueous solution does not contain a cryoprotectant, e.g., a cryoprotectant that is not the excipient. In some embodiments, the bacteria of the genus Mycobacterium are M. tuberculosis, M. smegmatis, M. bovis, or Bacillus Calmette-Guerin bacteria. In some embodiments, the methods further include formulating the dry powder in a pharmaceutical composition, e.g., for administration by inhalation or by injection after the powder is reconstituted in a liquid pharmaceutically acceptable carrier. In some embodiments, the methods further include formulating the dry powder as a vaccine, e.g., for administration by inhalation or by injection after the powder is reconstituted in a liquid pharmaceutically acceptable carrier. The invention also includes dry powders that include bacteria of the genus Mycobacterium that are produced by the new methods.

In another aspect, the invention includes vaccine compositions that include a dry powder with less than about 10% (e.g., less than about 8%, 5%, 4%, 3%, 2%, or 1%) water, e.g., free water, a cellular material, and at least 25% (e.g., at least 30%, 40%, 50%, 60%, 70%, 80%, 90%, 92%, 94%, 96%, 98%, 99%, or greater) of an excipient by dry weight. In some embodiments, the dry powder is produced by a method described herein. The vaccine composition can be formulated for parenteral or mucosal (e.g., oral or inhalation) administration. In some embodiments, the cellular material includes bacteria (e.g., bacteria of the genus Mycobacterium, e.g., M. tuberculosis, M. smegmatis, or Bacillus Calmette-Guerin), viruses, eukaryotic microbes, mammalian cells (e.g., red blood cells, stem cells, granulocytes, fibroblasts, or platelets), or membrane-bound organelles. Vaccine compositions can include one or more adjuvants. In some embodiments, the one or more adjuvants are spray-dried with the cellular material to form the dry powder. In some embodiments, the one or more adjuvants are blended with the dry powder following its production.

The invention also includes methods of immunization by administering to a subject (e.g., a human or animal) a vaccine composition that includes a dry powder described herein. In some embodiments, the dry powder is produced by a method described herein. The vaccine composition can be formulated for parenteral or mucosal (e.g., oral or inhalation) administration. In some embodiments, the subject is an infant, child, or adult. In some embodiments, the cellular material includes bacteria (e.g., bacteria of the genus Mycobacterium, e.g., M. tuberculosis, M. smegmatis, or Bacillus Calmette-Guerin), viruses, eukaryotic microbes, mammalian cells (e.g., red blood cells, stem cells, granulocytes, fibroblasts, or platelets), or membrane-bound organelles. Vaccine compositions for use in the methods of immunization can include one or more adjuvants.

In further aspects, the invention includes methods of storing a dry powder described herein by keeping the keeping the powder at a temperature above freezing, e.g., between 4° C. and 50° C. (e.g., between 4° C. and 40° C., between 4° C. and 30° C., between 4° C. and 20° C., between 4° C. and 10° C., between 10° C. and 50° C., between 10° C. and 40° C., between 10° C. and 30° C.) for a period of time of at least one day (e.g., at least one week, two weeks, three weeks, one month, two months, three months, four months, five months, six months, seven months, eight months, nine months, ten months, eleven months, one year, or longer). In some embodiments, the dry powder is kept at ambient temperature. In some embodiments, the dry powder is produced by a method described herein. In some embodiments, the dry powder is formulated as a pharmaceutical or vaccine composition.

In still further aspects, the invention includes methods of transporting a pharmaceutical or vaccine composition that includes a dry powder with less than about 10% (e.g., less than about 8%, 5%, 4%, 3%, 2%, or 1%) water, e.g., free water, a cellular material, and at least 25% (e.g., at least 30%, 40%, 50%, 60%, 70%, 80%, 90%, 92%, 94%, 96%, 98%, 99%, or greater) of an excipient by dry weight. The methods include producing the pharmaceutical or vaccine composition that includes a dry powder (e.g., a dry powder produced by a method described herein) and transporting the pharmaceutical or vaccine composition or vaccine composition at a temperature above freezing, e.g., between 4° C. and 50° C. (e.g., between 4° C. and 40° C., between 4° C. and 30° C., between 4° C. and 20° C., between 4° C. and 10° C., between 10° C. and 50° C., between 10° C. and 40° C., between 10° C. and 30° C.). In some embodiments, the pharmaceutical or vaccine composition is transported at ambient temperature.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram depicting a model of cellular material surrounded by water. R^{c} denotes the radius of the cell. c^{e}ₛ, C^{e}_{cp}, Cⁱₛ, and Cⁱ_{cp} indicate the concentrations of extracellular salt, extracellular cryoprotectant, intracellular salt, and intracellular cryoprotectant, respectively.

FIG. 2A is a two-dimensional depiction of parallel membranes.

FIG. 2B is a two-dimensional depiction of convex plateau borders.

FIG. 3 is an electron micrograph of the spray dried product of 80:20 Leu: M. smegmatis.

FIG. 4 is an electron micrograph of the spray dried product of 95:5 Leu: M. smegmatis.

FIG. 5 is a fluorescence micrograph of the spray dried product of 90:10 Leu: M. smegmatis. The M. smegmatis that were used expressed GFP, and show fluorescence in the micrograph.

FIG. 6 is an electron micrograph of 95:5 Leu: M. smegmatis after storage at 25° C. for one week.

FIG. 7 is a graph of numerical solutions describing relative cell volume (V/V₀) in a drying droplet under conditions: (a) greater amount of cryoprotectant inside the cell than outside the cell; (b) no cryoprotectant; (c) equal amounts of cryoprotectant inside and outside the cell.

FIG. 8 is a graph depicting the effect of glycerol and salt on viability of spray dried M. smegmatis as a result of similar osmotic stress.

FIG. 9 is a graph depicting the viability yield of M. smegmatis versus percentage of excipient (leucine) solution in spray dried powder.

FIG. 10 is a line graph depicting the viability yield of M. smegmatis over time at three storage conditions for the 50:50 leucine/smeg powders.

FIG. 11 is a line graph depicting the viability yield of M. smegmatis over time at three stability conditions for the 95:5 leucine/smeg powders. Results shown are the average of five experiments.

FIGS. 12A and 12B are line graphs depicting the viability yield of M. smegmatis over time at three stability conditions for the 95:5 leucine/smeg powders with or without monophospholipid A.

FIG. 13 is a graph depicting the viability yield of 95:5 and 50:50 Leu: M. smegmatis spray-dried in the presence of surfactants tyloxapol and Pluronic™-F68.

FIG. 14 is a line graph depicting the viability yield of M. bovis BCG over time at two storage conditions.

FIG. 15 is a micrograph of viable NIH 3T3 embryonic mouse fibroblast cells 1 month following spray drying.

FIG. 16 is a set of 20× phase contrast micrograph images of primary harvest rat cardiac fibroblasts at day 3 and day 8 following spray drying.

FIG. 17 is a set of 20× phase contrast micrograph images of NIH 3T3 embryonic mouse fibroblasts at day 3 and day 8 following spray drying.

### DETAILED DESCRIPTION

The invention relates to new compositions and methods for making dry cellular forms (DCF). These compositions and methods facilitate the production of dry forms of cellular material at large volumes and with good processing characteristics and cellular viability. In a preferred embodiment, the cellular materials are dried with initial excipient concentrations typically at least 50% (e.g., at least 60%, 70%, 80%, or 90%) by dry weight. However, in some instances the initial excipient concentrations can be as low as 25%. These excipients may be chosen or processed in such a fashion that the cellular materials are dried with cryoprotectants to reduce osmotic stress during the drying process.

The compositions and methods described herein can be used to dry any cellular material, for example, a cellular material relevant to pharmaceutical, agricultural, or food applications. “Cellular material” is used herein interchangeably with “membrane-bound material” and refers to material enclosed by a membrane composed of a lipid bilayer. Exemplary cellular materials include bacteria (e.g., Gram-negative and Gram-positive bacteria, and vaccine forms thereof), membrane-bound viruses (e.g., HIV), eukaryotic microbes (e.g., yeasts), mammalian cells (e.g., blood cells (e.g., umbilical cord blood cells), platelets, stem cells, granulocytes, fibroblasts, endothelial cells (e.g., vascular endothelial cells), muscle cells, skin cells, marrow cells, and other cells), membrane-bound organelles (e.g., mitochondria), liposomes, membrane-based bioreactors (Bosquillon et al., J. Control. Release, 99:357-367, 2004), and membrane-based drug delivery systems (Smith et al., Vaccine, 21:2805-12, 2003).

Further examples of cellular materials include membrane bound viruses (e.g., influenza virus, rabies virus, vaccinia virus, West Nile virus, HIV, HVJ (Sendai virus), hepatitis B virus (HBV), orthopoxviruses (e.g., smallpox and vaccinia virus), herpes simplex virus (HSV), and other herpesviruses). Other exemplary cellular materials include causative agents of viral infectious diseases (e.g., AIDS, AIDS Related Complex, chickenpox (varicella), common cold, cytomegalovirus infection, Colorado tick fever, Dengue fever, ebola hemorrhagic fever, epidemic parotitis, hand foot and mouth disease, hepatitis, herpes simplex, herpes zoster, human papilloma virus (HPV), influenza (flu), Lassa fever, measles, Marburg hemorrhagic fever, infectious mononucleosis, mumps, poliomyelitis, progressive multifocal leukencephalopathy, rabies, rubella, SARS, smallpox (Variola), viral encephalitis, viral gastroenteritis, viral meningitis, viral pneumonia, West Nile disease, and yellow fever), causative agents of bacterial infectious diseases (e.g., anthrax, bacterial meningitis, brucellosis, campylobacteriosis, cat scratch disease, cholera, diphtheria, epidemic typhus, gonorrhea, impetigo, legionellosis, leprosy (Hansen's disease), leptospirosis, listeriosis, Lyme disease, melioidosis, methicillin resistant Staphylococcus aureus (MRSA) infection, nocardiosis, pertussis (whooping cough), plague, pneumococcal pneumonia, psittacosis, Q fever, Rocky Mountain spotted fever (RMSF), salmonellosis, scarlet fever, shigellosis, syphilis, tetanus, trachoma, tuberculosis, tularemia, typhoid fever, typhus, and urinary tract infections), causative agents of parasitic infectious diseases (e.g., African trypanosomiasis, amebiasis, ascariasis, babesiosis, Chagas disease, clonorchiasis, cryptosporidiosis, cysticercosis, diphyllobothriasis, dracunculiasis, echinococcosis, enterobiasis, fascioliasis, fasciolopsiasis, filariasis, free-living amebic infection, giardiasis, gnathostomiasis, hymenolepiasis, isosporiasis, kala-azar, leishmaniasis, malaria, metagonimiasis, myiasis, onchocerciasis, pediculosis, pinworm infection, scabies, schistosomiasis, taeniasis, toxocariasis, toxoplasmosis, trichinellosis, trichinosis, trichuriasis, and trypanosomiasis), and causative agents of fungal infectious diseases (e.g., aspergillosis, blastomycosis, dandidiasis, doccidioidomycosis, dryptococcosis, histoplasmosis, and tinea pedis). Additionally, attenuated (e.g., auxotrophic) versions of the disease causing agents and related agents that can promote immunity against the disease causing agents (e.g., BCG and vaccinia) can be used in the methods described herein, e.g., for the production of vaccines (see, e.g., Sambandamurthy et al., Nat. Med., 9:9, 2002; Hondalus et al., Infect. Immun., 68:2888-98, 2000; and Sampson et al., Infect. Immun., 72:3031-37, 2004).

Excipients for use with the methods and compositions described herein include, but are not limited to, compatible carbohydrates, natural and synthetic polypeptides, amino acids, surfactants, polymers, or combinations thereof. Typical excipients will have a reflection coefficient less than 1.0 (e.g., less than 0.9, 0.8, 0.7, 0.6, 0.5, 0.4, 0.3, 0.2, or 0.1) for the membrane of the cellular material being dried (see, e.g., Adamski and Anderson, Biophys J., 44:79-90, 1983; and Janá\check{c}ek and Sigler, Physiol. Res., 49:191-195, 2000). Suitable carbohydrates include monosaccharides, such as galactose, D-mannose, sorbose, dextrose, and the like. Disaccharides, such as lactose, trehalose, maltose, sucrose, and the like can also be used. Other excipients include cyclodextrins, such as 2-hydroxpropyl-β-cyclodextrin; and polysaccharides, such as raffinose, maltodextrins, dextrans, and the like; and alditols, such as mannitol, xylitol, sorbitol, and the like. Suitable polypeptides include the dipeptide aspartame. Suitable amino acids include any of the naturally occurring amino acids that form a powder under standard pharmaceutical processing techniques and include the non-polar (hydrophobic) amino acids and the polar (uncharged, positively charged and negatively charged) amino acids, such amino acids are generally regarded as safe (GRAS) by the FDA. Representative examples of non-polar amino acids include alanine, isoleucine, leucine, methionine, phenylalanine, proline, tryptophan, and valine. Representative examples of polar, uncharged amino acids include cysteine, glutamine, serine, threonine, and tyrosine. Representative examples of polar, positively charged amino acids include arginine, histidine, and lysine. Representative examples of negatively charged amino acids include aspartic acid and glutamic acid. Suitable synthetic organic polymers include poly[1-(2-oxo-1-pyrrolidinyl)ethylene], i.e., povidone or PVP.

### Dried Compositions

Typically, cellular materials are dried with relatively small quantities of excipients, often involving freezing. In the absence of freezing, the resultant powders tend to contain a significant amount of water, owing to the fact that cellular materials cannot, barring freezing, be dried below a given water content (e.g., approximately 40% water by weight), and still remain active. Dried powders with good processing and stability properties require typically less than 10% and preferably less than 5% water by weight. This is because larger water fractions lead to significant capillary forces between particles of the powder and thus aggregation of the powder. To achieve DCF with good powder processing and stability characteristics therefore involves spray drying with a large amount of excipient. Specifically, to achieve dry powders with total water content less than 10% or 5%, at least 25% by weight (e.g., at least 30%, 40%, 50%, 60%, 70%, 80%, 90%, 92%, 94%, 96%, 98%, 99%, or greater) of excipient should be dried with the cellular form, resulting in a dry powder that contains a relatively small weight fraction of cellular material, which, while retaining enough water to remain active, does not present so much water to the powder as to harm the overall processing properties of the powder.

Spray drying is a standard process used in the food, pharmaceutical, and agricultural industries. In spray drying, moisture is evaporated from an atomized feed (spray) by mixing sprayed droplets with a drying medium (e.g., air or nitrogen). This process dries the droplets of their volatile substance and leaves non-volatile components of “dry” particles that are of a size, morphology, density, and volatile content controlled by the drying process. The mixture being sprayed can be a solvent, emulsion, suspension, or dispersion. Many factors of the drying process can affect the properties of the dry particles, including the type of nozzle, drum size, flow rate of the volatile solution and circulating gas, and environmental conditions (Sacchetti and Van Oort, Spray Drying and Supercritical Fluid Particle Generation Techniques, Glaxo Wellcome Inc., 1996).

Typically, the process of spray drying involves four processes, dispersion of a mixture in small droplets, mixing of the spray and a drying medium (e.g., air), evaporation of moisture from the spray, and separation of the dry product from the drying medium (Sacchetti and Van Oort, Spray Drying and Supercritical Fluid Particle Generation Techniques, Glaxo Wellcome Inc., 1996).

The dispersion of the mixture in small droplets greatly increases the surface area of the volume to be dried, resulting in a more rapid drying process. Typically, a higher energy of dispersion leads to smaller droplets obtained. The dispersion can be accomplished by any means known in the art, including pressure nozzles, two-fluid nozzles, rotary atomizers, and ultrasonic nozzles (Hinds, Aerosol Technology, 2^{nd} Edition, New York, John Wiley and Sons, 1999). In some embodiments, the mixture is sprayed at a pressure less than 200 psi.

Following the dispersion (spraying) of the mixture, the resultant spray is mixed with a drying medium (e.g., air). Typically, the mixing occurs in a continuous flow of heated air. The hot air improves heat transfer to the spray droplets and increases the rate of evaporation. The air stream can either be exhausted to the atmosphere following drying or recycled and reused. Air flow is typically maintained by providing positive and/or negative pressure at either end of the stream (Sacchetti and Van Oort, Spray Drying and Supercritical Fluid Particle Generation Techniques, Glaxo Wellcome Inc., 1996).

When the droplets come into contact with the drying medium, evaporation takes place rapidly due to the high specific surface area and small size of the droplets. Based on the properties of the drying system, a residual level of moisture may be retained within the dried product (Hinds, Aerosol Technology, 2^{nd} Edition, New York, John Wiley and Sons, 1999).

The product is then separated from the drying medium. Typically, primary separation of the product takes place at the base of the drying chamber, and the product is then recovered using, e.g., a cyclone, electrostatic precipitator, filter, or scrubber (Masters et al., Spray Drying Handbook. Harlow, UK, Longman Scientific and Technical, 1991).

The properties of the final product, including particle size, final humidity, and yield depend on many factors of the drying process. Typically, parameters such as the inlet temperature, air flow rate, flow rate of liquid feed, droplet size, and mixture concentration are adjusted to create the desired product (Masters et al., Spray Drying Handbook, Harlow, UK, Longman Scientific and Technical, 1991).

The inlet temperature refers to the temperature of the heated drying medium, typically air, as measured prior to flowing into the drying chamber. Typically, the inlet temperature can be adjusted as desired. The temperature of the drying medium at the product recovery site is referred to as the outlet temperature, and is dependent on the inlet temperature, drying medium flow rate, and properties of the sprayed mixture. Typically, higher inlet temperatures provide a reduction in the amount of moisture in the final product (Sacchetti and Van Oort, Spray Drying and Supercritical Fluid Particle Generation Techniques, Glaxo Wellcome Inc., 1996).

The air flow rate refers to the flow of the drying medium through the system. The air flow can be provided by maintaining positive and/or negative pressure at either end or within the spray drying system. Typically, higher air flow rates lead to a shorter residence time of the particles in the drying device (i.e., the drying time) and lead to a greater amount of residual moisture in the final product (Masters et al., Spray Drying Handbook, Harlow, UK, Longman Scientific and Technical, 1991).

The flow rate of the liquid feed refers to the quantity of liquid delivered to the drying chamber per unit time. The higher the throughput of the liquid, the more energy is needed to evaporate the droplets to particles. Thus, higher flow rates lead to lower output temperatures. Typically, reducing the flow rate while holding the inlet temperature and air flow rate constant reduces the moisture content of the final product (Masters et al., Spray Drying Handbook, Harlow, UK, Longman Scientific and Technical, 1991).

The droplet size refers to the size of the droplets dispersed by the spray nozzle. Typically, smaller droplets provide lower moisture content in the final product with smaller particle sizes (Hinds, Aerosol Technology, 2^{nd} Edition, New York, John Wiley and Sons, 1999).

The concentration of the mixture to be spray dried also influences the final product. Typically, higher concentrations lead to larger particle sizes of the final product, since there is more material per sprayed droplet (Sacchetti and Van Oort, Spray Drying and Supercritical Fluid Particle Generation Techniques, Glaxo Wellcome Inc., 1996).

Systems for spray drying are commercially available, for example, from Armifield, Inc. (Jackson, N.J.), Brinkmann Instruments (Westbury, N.Y.), BUCHI Analytical (New Castle, Del.), Niro Inc (Columbia, Md.), Sono-Tek Corporation (Milton, N.Y.), Spray Drying Systems, Inc. (Randallstown, Md.), and Labplant, Inc. (North Yorkshire, England).

The final moisture content of the spray dried powder can be determined by any means known in the art, for example, by thernogravimetric analysis. The moisture content is determined by thermogravimetric analysis by heating the powder, and measuring the mass lost during evaporation of moisture (Maa et al., Pharm. Res., 15:5, 1998). Typically, for a sample that contains cellular material (e.g., bacteria), the water will be evaporated in two phases. The first phase, referred to as free water, is primarily the water content of the dry excipient. The second phase, referred to as bound water, is primarily the water content of the cellular material. Both the free and bound water can be measured to determine if the powder contains a desired moisture content in either the excipient or cellular material (Snyder et al., Analytica Chimica Acta, 536:283-293, 2005).

### Reducing Osmotic Stress During Spray Drying

The excipients introduced into the cellular solution to be spray dried might be chosen and/or introduced in such a way as to minimize the overall osmotic stress on the membranes of the cellular materials and therefore to maintain activity. While it is important, for reasons described above, to retain a desired mass fraction of excipient relative to the mass fraction of cellular material, the nature of these excipients, and the means in which they are introduced prior to spray drying, can be important and even critical for cell viability.

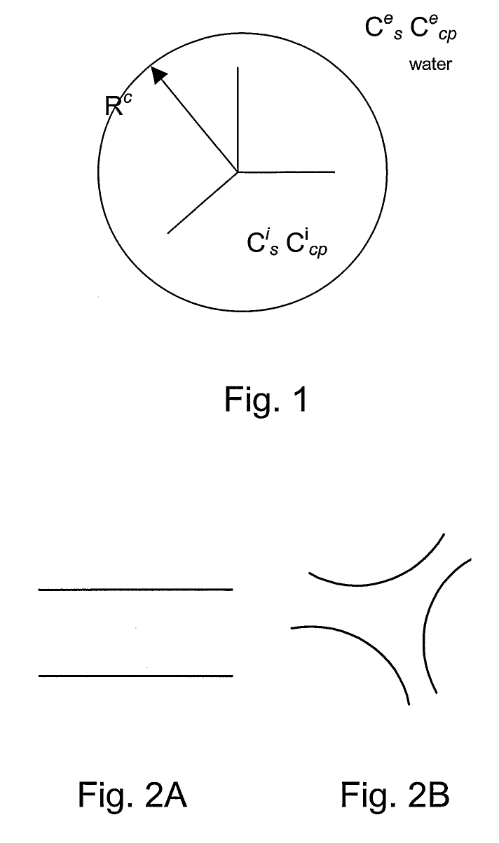

For cellular material, the drying of droplets in a spray drying drum may be viewed as analogous to the freezing of an organism in a standard cryopreservation process, as shown in FIG. 1 (James, “Maintenance of Parasitic Protozoa by Cryopreservation,” Maintenance of Microorganisms, Academic Press, London, 1984.).

When a droplet containing an organism evaporates, the concentration of salt (C^{e}ₛ) in the droplet (and outside the cell) will increase relative to the salt concentration in the organism (Cⁱₛ). The reason is that the cell membrane is impermeable to the transfer of salt, while it is relatively permeable to the transfer of water. The consequence is that droplet drying increases the salt concentration in the evaporating droplet and creates osmotic stresses on the cell membrane (caused by the imbalance of the salt concentration on either side of the membrane), which cause water to be pushed out of the cell. This dehydration process can be thought of as the membrane's attempt to mechanically reduce the osmotic stress by eliminating the salt concentration imbalance (Batycky et al., Phil. Trans. Roy. Soc. Lond., A355:2459-88, 1997).

The “dehydration” of cellular material during droplet evaporation is essentially the same process that arises when cellular material undergoes freezing. To avoid excessive dehydration, which can, as described above, lyse the cellular material, techniques associated with the field of cryopreservation, namely the use of cryoprotectants and the control of freezing and thawing cycles, have been developed. Cryoprotectants are pharmacologically inert substances that permeate the cell membrane at a rate slower than water but faster than salt. As these techniques are relevant to methods of spray drying cellular material, they are briefly reviewed below (Karlsson and Toner, Biomaterials, 17: 243-256, 1996).

First, given the membrane's semipermeability to cryoprotectants, cryoprotectants deliver an osmotic pressure on the membrane—one that is proportional to cryoprotectant concentration and, for the most successful cryoprotectants one that is very near to the osmotic pressure delivered by salt at equivalent concentration. This means that cell membranes that are immersed in aqueous media containing cryoprotectant of similar magnitude of impermeable salt concentration will tend to experience osmotic stress and non-isotonic conditions that are significantly influenced by the presence of cryoprotectant material. Diffusion of cryoprotectant across the membrane therefore provides a means for off setting osmotic stresses even in the circumstances where salt concentrations are unequal on either side of the membrane. For this reason, cryoprotectants provide a mechanism for diffusing osmotic stresses. Suitable cryoprotectants for use with the new methods include, but are not limited to, dimethyl sulfoxide, ethylene glycol, propylene glycol, and glycerol (Chesne and Guillouzo, Cryobiology, 25:323-330, 1988.). In some embodiments, cryoprotectants are excluded from the dried mixture.

In cryopreservation protocols, cryoprotectants are added to suspensions of cellular material at a concentration (C^{e}_{cp}) that is significant relative to salt concentration. It is noteworthy that this addition can be controlled so as not to subject the cells to excessive osmotic stress, i.e., the cryoprotectant can be added at a rate that is sufficiently slow so that cryoprotectants can diffuse across the cell membrane and not dehydrate the cell. Then, during freezing—which leads to ice formation outside of the cell owing to natural cryoprotectants within the cell, thus increasing salt concentration outside the cell—the cryoprotectant is able to diffuse across the cell membrane and raise the internal cellular concentration, which increases the internal concentration of cryoprotectant (Cⁱ_{cp}). This relieves the osmotic pressure on the cell membrane, especially if the freezing occurs at a slow enough rate. In this way, cryoprotectants contribute to preservation of cell viability, explaining its use for preserving blood, sperm, and other useful cells (Karlsson and Toner, Biomaterials, 17: 243-256, 1996).

Notwithstanding its analogy to cryopreservation, spray drying provides a distinct advantage for cellular material that is especially relevant for large scale use. Cryopreservation of cells is challenged by large volumes of cellular suspensions in that the mass transfer kinetic requirements (involved in adding or removing cryoprotectant, and freezing cells) are very different on the cellular and suspension scale, when the latter is far larger than the former. This may be one of the reasons why the freezing of blood by standard methods of cryopreservation does not easily apply to freezing of whole organs. Spray drying automatically divides the cellular suspension into small volumes (i.e., droplets) that can be loosely viewed as small cryopreservation units. Scale-up does not require a significant increase in the volume of the sprayed droplets: rather, scale up is achieved by increasing the size of the spray drying vessel, increasing the flow of suspension through the nozzle, and other standard scale up measures.

Spray drying can thus provide a method for producing large volumes of DCF with greater activity than would otherwise be achieved through the techniques of cryopreservation and lyophilization.

In the following, a theoretical formalism is described that provides rules for spray drying cellular forms in a way that minimizes membrane stress and therefore maximizes viability. The methods rely on the use of cryoprotectants and the control of standard spray drying parameters, e.g., solvent type, inlet gas temperature, and spray drying nozzle dimensions and speed of rotation (droplet size).

The methods determine the rate at which sprayed droplets can be dried within a heated environment such that, in the presence of cryopreservative agents, the membrane radius of suspended material can be modulated. Thus, the membrane can be prevented from shrinking below R^{c}ₘᵢₙ or expanding above R^{c}ₘₐₓ. For the purpose of illustration in the case of R^{c}ₘᵢₙ, all suspended material will not shrink below a critical radius (R^{c}_{cri}) as a consequence of osmotically driven dehydration. In cases of rigid cellular walls, this condition can straightforwardly be equated with a critical stress that leads to deactivation. First, the idealized geometry and concentrations within the problem are considered, followed by a consideration of the kinematics in two limiting conditions. After this, the fluid dynamic and mass transfer equations are developed to describe the rate of change of cell radii as a function of parameters of the system.

One can imagine a suspension of cells where, for the sake of illustration, cells are spheres with an equilibrium radius R^{c}ₒ. Within the cells, there are salts and cryoprotectants at concentrations Cⁱₛ and cⁱ_{cp} inside the cells and outside the cell in concentrations of C^{e}ₛ and C^{e}_{cp}.

Upon spray drying, individual droplets of suspended material are formed. Here, it is assumed that the cells remain homogeneously distributed in the spray solution and spray process and are therefore at equal concentration in the individual sprayed droplets. The flow rate, which can be physically controlled during spray drying can be explicitly solved for:

$$a = \frac{N}{{n}_{cells} {t}_{o}} \qquad ( 1 )$$

where a is the rate of droplets created per unit of time, n_{cells} is the number of cells suspended in each individual sprayed droplet, N is the total number of cells in the volume, and tₒ is the amount of time required to spray the volume Vₒ.

The volume fraction of cells in the suspension to be sprayed will be referred to as φₒ where

$${φ}_{o} = \frac{total cell volume}{suspension volume} = \frac{{NR}_{o}^{C}}{{V}_{o}} \qquad ( 2 )$$

and N is the total number of cells in the suspension volume φₒ.

These droplets are assumed to possess a uniform radius R^{d}ₒ, such that the fraction of cellular material can be expressed as

$${φ}_{o} = {{n}_{cells} ( \frac{{R}_{o}^{c}}{{R}_{o}^{d}} )}^{3} \qquad ( 3 )$$

where n is the number of cells suspended in each individual sprayed droplet.

Assuming homogeneity, the four concentrations C^{e}ₛ, C^{e}_{cp}, Cⁱₛ, Cⁱ_{cp} measured in the original suspension are equal to the initial concentration of salt and cryoprotectant within the cell of each sprayed droplet. These concentrations will change with time based upon changes in the droplet diameter and cell diameter, given that the absolute number of moles of salt and cryoprotectant must be conserved within each droplet.

Let xⁱₛ and x^{e}ₛ, and xⁱ_{cp} and x^{e}_{cp}, denote the moles of salt and cryoprotectant respectively within the exterior and interior of the cells following their dispersion within the individual droplets. This gives:

$$\begin{matrix}{C}_{s}^{i} = \frac{{x}_{s}^{i}}{4 / 3 π {R}^{{c}^{3}} - {V}_{excluded}^{c}} & ( 4 ) \\ {C}_{cp}^{i} = \frac{{x}_{cp}^{i}}{4 / 3 π {R}^{{c}^{3}} - {V}_{excluded}^{c}} & ( 5 ) \\ {C}_{s}^{e} = \frac{{x}_{s}^{e}}{4 / 3 π {R}^{{d}^{3}} ( 1 - φ )} = \frac{{x}_{s}^{e}}{4 / 3 π [ {R}^{{d}^{3}} - {n}_{cells} {R}^{{c}^{3}} ]} & ( 6 ) \\ {C}_{cp}^{e} = \frac{{x}_{cp}^{e}}{4 / 3 π {R}^{{d}^{3}} ( 1 - φ )} = \frac{{x}_{cp}^{e}}{4 / 3 π [ {R}^{{d}^{3}} - {n}_{cells} {R}^{{c}^{3}} ]} & ( 7 )\end{matrix}$$

Here V^{c}_{excluded} is the volume of each individual cell into which salt and/or cryoprotectant is unable to partition, and will be considered a constant with respect to time. The parameters xⁱₛ and x^{e}ₛ (representing the moles of salt inside and outside of the cell) are also constant with respect to time due to impermeability of salt through the membrane. The sole time variables in these expressions then become R^{c} and R^{d}, and the moles of cryoprotectant inside and outside of the cell are xⁱ_{cp} and x^{e}_{cp}.

Each individual droplet will evaporate in the spray drying drum at a rate dependent upon the external conditions, droplet size, droplet volatility etc. Initially, the individual cells will be on average far removed from each other given the initial dilute nature of the suspension (φₒ<<1). Over time, the cells will increasingly come into intimate contact, such that one can imagine two limiting cases:

Here, φ(t)<<1 during the drying process. In this case, it is assumed that each individual cell is isolated and responding to evolving salt and cryoprotectant concentration (and consequently osmotic stress) as if it were suspended within an infinite bath. The symmetry of the problem (see below for mass transfer considerations) is such that the droplets and cells all contract (or expand) radially. Therefore, considering FIG. 1, the velocity profile created within and around the individual cell owing to the osmotic stresses and not due to fluid motion can be expressed as:

v=ιᵣvᵣ(t) (8)

where ιᵣ is the unit vector directed along the coordinate r in a spherical coordinate system originating at the center of the cell and νᵣ(t) is the magnitude of the radial velocity.

Moreover, given that the cell and droplet fluids are incompressible.

$$\begin{matrix}∇ \cdot v = 0 \\ or & ( 9 ) \\ \frac{\partial {v}_{r}}{\partial r} = 0. & ( 10 )\end{matrix}$$

Since the radial velocity at the center of the cell must be zero, it is concluded that

v=0 (11)

everywhere. This conclusion implies that any radial motion of the cell membrane must be “non-material,” meaning that the membrane motion is not equal to the mass average motion of the contiguous fluid.

Case 1 is therefore a problem wherein the evolution of individual cells within the droplet is diffusively driven.

In the limit of φₒ→1, individual cells within the drying droplet come within extremely close contact. The evolution of the cell membranes, as consequence of osmotic stress, is determined within an environment where cell membranes either flatten next to the neighboring cells or curve in a convex fashion in the vicinity of so-called “Plateau borders.” These membrane circumstances are shown in FIG. 2.

Several of the basic assumptions in Case 1 are no longer valid in Case 2. First, given the intimate contact of the cells and mass transfer resistance in the “contiguous” phase of the droplet caused by the excluded volume of the cells, increases in salt and cryoprotectant concentrations in the external or continuous phase cannot be expected to be instantaneous relative to the water transport across the cell membrane. This means that as the droplet volume continues to diminish, the concentration of salt and cryoprotectant in the periphery of the droplet will increase significantly relative to the concentration near the center of the droplet, thus cells near the periphery of the droplet will undergo high osmotic stress while cells in the center will go through little or no osmotic stress. The objective of minimizing each cell's radial expansion or contraction during the drying process then has ambiguous meaning, since each cell will experience a variety of conditions over time. Either the object in Case 2 is to minimize cell dilatation for the most vulnerable cells, those at the periphery, or to salvage the greatest number of cells within the droplet given reasonable time constraints on the drying. (Note that the ultimate drying restrictions required to minimize cell death at the periphery might in the limit require drying of infinite slowness.)

For the purpose of this analysis, the remaining considerations will remain focused exclusively on Case 1.

Two significant mass transfer problems can be identified for Case 1. The first relates to the mass transfer of salt and cryoprotectant within the drying droplet given that the concentration of salt and cryoprotectant increases uniformly within the drying droplet as a function of time. Owing to the diluteness of the cell suspension, the droplet drying problem can be considered separately. This latter problem is that of a spherical water droplet drying in a continuum of hot air.

The mass transfer problem of a spherical cell within an unbounded environment where the external salt and cryoprotectant concentration suddenly change uniformly has been previously solved by Batycky et al. (1997). In their analysis, the cellular fluid is described as a continuum, where the salt and cryoprotectant concentration within the cell is viewed as homogenized, or specially averaged, over the cytosolic fluid and internal organelles. Using the standard definition for osmotic pressure on the membrane, the Reynolds Transport Theorem and a Darcy law description of water permeability through the membrane, it can be shown that the velocity of the membrane is,

$$\begin{matrix}U = \frac{d {R}_{o}^{c}}{d t} \\ = - {L}_{p} {R}_{gas} T [ ( {C}_{s}^{e} - {C}_{s}^{l} {\vert}_{R = {R}_{c} ( t )} ) + σ ( {C}_{cp}^{e} - {C}_{cp}^{l} {\vert}_{R = R ( t )} ) ]\end{matrix} \qquad ( 12 )$$

where Lₚ is the hydraulic permeability of the membrane (m/s·atm) and σ, known as the reflection coefficient (0<σ<1), represents the fraction by which the permeability of the membrane to cryoprotectant is diminished relative to salt.

The time rate of change of salt and cryoprotectant concentration within the cell at the membrane can be determined by the solution to the associated mass transfer conservation equations. Notwithstanding the high concentration of salt and cryopreservation agent within the cell, Fickian diffusion is assumed for constant salt and cryoprotectant. Following Batycky et al. (1997) and incorporating results of Edwards and Davis (Chem. Eng. Sci., 50:1441-54, 1995), these diffusivities are expressed as course-scale coefficients ( D̅ₛ̅*̅, D̅_̅{̅c̅p̅}̅*̅) that reflect the presence of organelles within the cell.

The governing differential equations for salt concentration can be expressed in Batycky et al. (1997):

$$\begin{matrix}\frac{\partial \overline{{C}_{s}^{i}}}{\partial t} = \frac{1}{{r}^{2}} \frac{\partial }{\partial r} ( {D}_{s}^{*} {r}^{2} \frac{\partial \overline{{C}_{s}^{i}}}{\partial r} ) & ( 13 ) \\ \overline{{C}_{s}^{i}} = finite , ∀ r = 0 , t & ( 14 ) \\ \overline{{D}_{s}^{*}} \frac{\partial \overline{{C}_{s}^{i}}}{\partial r} + \frac{d {R}^{c} ( t )}{d t} {C}_{s}^{i} = 0 , ∀ r = R ( t ) , t & ( 15 )\end{matrix}$$

given initial conditions

Cₛⁱ=Cₛⁱ(0), at t=0, where R^{c}(t)=Rᵢ, at t=0 (16)

In the above equation, Cₛⁱ and C̅ₛ̅ⁱ̅ are related by

$$\overline{{C}_{s}^{i}} = {C}_{s}^{i} ( 1 - \frac{{V}_{excluded}^{c}}{\frac{4}{3} π {{R}^{c} ( t )}^{3}} ) \qquad ( 17 )$$

These equations can be solved to yield:

$$\begin{matrix}\overline{{C}_{s}^{i}} = \frac{{x}_{s}^{i}}{\frac{4}{3} π {{R}^{c} ( t )}^{3}} & ( 18 ) \\ {C}_{s}^{i} = \frac{{x}_{s}^{i}}{\frac{4}{3} π {{R}^{c} ( t )}^{3} - {V}_{excluded}^{c}} & ( 19 )\end{matrix}$$

The governing differential equations for the cryoprotectant concentration can be expressed in Batycky et al. (1997):

$$\frac{\partial \overline{{C}_{cp}^{i}}}{\partial t} = \frac{1}{r} \frac{\partial }{\partial r} ( \overline{{D}_{cp}^{*}} {r}^{2} \frac{\partial \overline{{C}_{cp}^{i}}}{\partial r} ) , \qquad ( 20 )$$

subject to boundary conditions,

$$\begin{matrix}\overline{{C}_{cp}^{i}} = finite , ∀ r = 0 , t , & ( 21 ) \\ \overline{{D}_{cp}^{*}} = \frac{\partial \overline{{C}_{cp}^{i}}}{\partial r} + \frac{d {R}^{c} ( t )}{d t} {C}_{cp}^{i} = {P}_{cp} ( {C}_{cp}^{e} - {C}_{cp}^{i} ) , ∀ r = ( {R}^{c} ( t ) , t ) & ( 22 )\end{matrix}$$

with initial conditions of

C_{cp}ⁱ=C_{cp}ⁱ(0), at t=0 (23)

R^{c}(t)=Rₒ^{c}, at t=0 (24)

and the relations where

C̅_̅{̅c̅p̅}̅ⁱ̅=C_{cp}ⁱ(1−θ+κα+Kθ), ∀r, t (25)

where θ is the osmotically inactive fraction of the cell (organelles), κ=Henry's law absorption coefficient, α the specific surface area of the organelles, and K the partition coefficient into the organelles.

Solving these equations with Eq. (14) yields (Batycky et al. 1997)

$$- \frac{1}{{L}_{p} {R}_{gas} T} \frac{d {R}^{c} ( t )}{d t} = {C}_{s}^{e} - \frac{{x}_{s}^{i}}{\frac{4}{3} π {{R}^{c} ( t )}^{3} - {V}_{excluded}} + σ [ {C}_{cp}^{e} - {C}_{cp}^{i} ( 0 ) ] 2 {\sum }_{n = 1}^{∞} \frac{{sin}^{2} ( {λ}_{n} ) - {λ}_{n} sin ( {λ}_{n} ) cos ( {λ}_{n} )}{{λ}_{n}^{2} - {λ}_{n} sin ( {λ}_{n} ) cos ( {λ}_{n} )} {e}^{- {λ}_{n}^{2} \overline{{D}_{cp}^{i}} t / {R}^{c} ( t} \qquad ( 26 )$$

subject to the initial conditions

R^{c}(t)=Rₒ^{c}, at t=0 (27)

Here λₙ are eigenvalues of the non-zero roots of the transcendental equation

βλₙ=tan(λₙ) (28)

with Pₛₚ the rate of semipermeable solute entry into the cell and the coefficient β defined as

$$β = {( 1 - \frac{{P}_{sp} {R}^{c} ( t )}{\overline{{D}_{sp}^{*}} ( 1 - θ + κ α + κ θ )} )}^{- 1} \qquad ( 29 )$$

Note that while λₙ are essentially constant over the rapid time scale of diffusion they slowly change in time over the time scale of cell membrane expansion. Equation (28) relates the cell radius R^{c}(t) to the external salt and cryopreservation concentration which in turn depend on the rate of evaporation of the droplet. This relationship is described below.

Many researchers have examined a spherical droplet drying in a gas phase particularly when convection effects in the gas are neglected. Evaporation within a spray dryer is dependent upon the governing rate of evaporation and residence time of evaporation. The residence time is a function of spray-air movement in the dryer. In the case of droplets moving relative to the surrounding air, flow conditions around the moving droplet influence evaporation rate. In this case, the droplet is completely influenced by air flow where the relative velocity between the air and the droplet is very low. According to boundary layer theory, the evaporation rate for a droplet moving with zero relative velocity is identical to evaporation in still-air conditions. Thus, the evaporation of the droplet via spray drying is modeled as a similar mechanism for evaporation in still-air conditions.

Both experimentally and theoretically, the general relationship observed between droplet radius and controlling parameters of the spray drying process is given by (Masters, 1991, Spray Drying Handbook, Longman Scientific and Technical, Harlow, UK):

$$d t = - \frac{{λρ}_{1} D}{{K}_{d} L M T D} d D \qquad ( 30 )$$

with D=2R_{c}, K_{d} the average thermal conductivity of the gaseous film surrounding an evaporating droplet, ρ₁ the density of the gas phase, λ the latent heat of vaporization of the droplet, and LMTD the logarithmic mean temperature difference defined by

$$L M T D = \frac{Δ {T}_{0} - Δ {T}_{1}}{2 \cdot 303 {log}_{10} ( Δ {T}_{0} / Δ {T}_{1} )} \qquad ( 31 )$$

where ΔTₒ and ΔT₁ are the initial and final temperature differences between the droplet and the gas phase.
Integration of (30) yields

$$\begin{matrix}{R}^{d} ( t ) = \sqrt{kt + {R}_{o}^{{d}^{2}}} \\ where & ( 32 ) \\ k = - \frac{{K}_{d} L M T D}{{λρ}_{1}} & ( 33 )\end{matrix}$$

Substitution of (32) into (6) and (7) relates the instantaneous concentrations of salt and cryoprotectant to droplet evaporation parameters:

$$\begin{matrix}\begin{matrix}{C}_{s}^{e} = \frac{{x}_{s}^{e}}{\frac{4}{3} {π ( kt + {R}_{o}^{{d}^{2}} )}^{3 / 2} ( 1 - φ )} \\ = \frac{{x}_{cp}^{e}}{\frac{3}{4} π [ {( kt + {R}_{o}^{{d}^{2}} )}^{3 / 2} - {{n}_{cells} ( {R}^{c} ( t ) )}^{3} ]}\end{matrix} & ( 34 ) \\ \begin{matrix}{C}_{cp}^{e} = \frac{{x}_{cp}^{e}}{\frac{4}{3} {π ( kt + {R}_{o}^{{d}^{2}} )}^{3 / 2} ( 1 - φ )} \\ = \frac{{x}_{cp}^{e}}{\frac{4}{3} π [ {( kt + {R}_{o}^{{d}^{2}} )}^{3 / 2} - {{n}_{cells} ( {R}^{c} ( t ) )}^{3} ]}\end{matrix} & ( 35 )\end{matrix}$$

The method for spray drying can be expressed in terms of the following differential equation:

$$- \frac{1}{{L}_{p} {R}_{gas} T} \frac{d {R}^{c} ( t )}{d t} = \frac{{x}_{s}^{e}}{\frac{4}{3} π [ {( kt + {R}_{o}^{{d}^{2}} )}^{3 / 2} - {{n}_{cells} ( {R}^{c} ( t ) )}^{3} ]} - \frac{{x}_{s}^{i}}{\frac{4}{3} π {{R}^{c} ( t )}^{3} - {V}_{excluded}} + σ [ \frac{{x}_{cp}^{e}}{\frac{4}{3} π [ {( kt + {R}_{o}^{{d}^{2}} )}^{3 / 2} - {{n}_{cells} ( {R}^{c} ( t ) )}^{3} ]} - {C}_{cp}^{i} ( 0 ) ] 2 {\sum }_{n = 1}^{∞} \frac{{sin}^{2} ( {λ}_{n} ) - {λ}_{n} sin ( {λ}_{n} ) cos ( {λ}_{n} )}{{λ}_{n}^{2} - {λ}_{n} sin ( {λ}_{n} ) cos ( {λ}_{n} )} {e}^{- {λ}_{u}^{2} \overline{{D}_{cp}^{*}} t /} \qquad ( 36 )$$

By evaluating the above equation, one can determine the conditions for the inlet and outlet gas temperatures of the spray dryer (i.e., ΔT), the nozzle type and speed of rotation for droplet size (R^{d}), the type of solvent (λ), and the type of cryoprotectant (Lₚ) necessary to minimize stress, permit the maintenance of Rₘᵢₙ^{c}<R^{c}(t)<Rₘₐₓ^{c}, or to maximize stress on suspended membrane-bound material. These rules find their parallel in rules of cryopreservation for rates of freezing and thawing of cells.

### Pharmaceutical Compositions

The dry cellular forms described herein, e.g., produced with the new compositions or by the new methods, can be prepared as pharmaceutical compositions, e.g., vaccine compositions. The cellular material may be spray dried with various pharmaceutically acceptable diluents, fillers, salts, buffers, stabilizers, solubilizers, and other materials well known in the art to make a pharmaceutical powder. Alternately, following spray drying, the product may be formulated with at least one of various pharmaceutically acceptable diluents, fillers, salts, buffers, stabilizers, solubilizers, adjuvants and other materials well known in the art to make a pharmaceutical composition, e.g., a pharmaceutical powder. The term “pharmaceutically acceptable” means a nontoxic material that does not interfere with the effectiveness of the biological activity of the active ingredient(s). The characteristics of the composition can depend on the route of administration. In some embodiments, the compositions can be stored at a controlled temperature prior to administration.

Administration of a pharmaceutical composition (e.g., a pharmaceutical composition containing a dry cellular form) can be carried out in a variety of conventional ways, such as inhalation, oral ingestion, or cutaneous, subcutaneous, or intravenous injection. Administration by inhalation is preferred. In some embodiments, the compositions are administered as a vaccine.

The dry cellular forms can be formulated for inhalation using a medical device, e.g., an inhaler (see, e.g., U.S. Pat. Nos. 6,102,035 (a powder inhaler) and 6,012,454 (a dry powder inhaler). The inhaler can include separate compartments for the active compound at a pH suitable for storage and another compartment for a neutralizing buffer, and a mechanism for combining the compound with a neutralizing buffer immediately prior to atomization. In one embodiment, the inhaler is a metered dose inhaler.

The three common systems used to deliver drugs locally to the pulmonary air passages include dry powder inhalers (DPIs), metered dose inhalers (MDIs) and nebulizers. MDIs, used in the most popular method of inhalation administration, may be used to deliver medicaments in a solubilized form or as a dispersion. Typically MDIs comprise a Freon or other relatively high vapor pressure propellant that forces aerosolized medication into the respiratory tract upon activation of the device. Unlike MDIs, DPIs generally rely entirely on the inspiratory efforts of the patient to introduce a medicament in a dry powder form to the lungs. Nebulizers form a medicament aerosol to be inhaled by imparting energy to a liquid solution. Direct pulmonary delivery of drugs during liquid ventilation or pulmonary lavage using a fluorochemical medium has also been explored. These and other methods can be used to deliver a dry cellular form. Exemplary inhalation devices are described in U.S. Pat. Nos. 6,732,732 and 6,766,799.

The compositions may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount. Capsules and cartridges for use in an inhaler or insufflator may be formulated containing dry cellular form.

Although not necessary, delivery enhancers such as surfactants can be used to further enhance pulmonary delivery. A “surfactant” as used herein refers to a compound having hydrophilic and lipophilic moieties that promote absorption of a drug by interacting with an interface between two imniscible phases. Surfactants are useful with dry particles for several reasons, e.g., reduction of particle agglomeration, reduction of macrophage phagocytosis, etc. When coupled with lung surfactant, a more efficient absorption of the compound can be achieved because surfactants, such as DPPC, will greatly facilitate diffusion of the compound. Surfactants are well known in the art and include, but are not limited to, phosphoglycerides, e.g., phosphatidylcholines, L-alpha-phosphatidylcholine dipalmitoyl (DPPC) and diphosphatidyl glycerol (DPPG); hexadecanol; fatty acids; polyethylene glycol (PEG); polyoxyethylene-9; auryl ether; palmitic acid; oleic acid; sorbitan trioleate (Span™ 85); glycocholate; surfactin; poloxomer; sorbitan fatty acid ester; sorbitan trioleate; tyloxapol; and phospholipids.

In another aspect, the dry cellular forms can be formulated with a pharmaceutically-acceptable carrier having a particle size that is not respirable, i.e., is of such a size that it will not be taken into the lungs in any significant amount. This formulation can be a uniform blend of smaller particles of the dry cellular form (e.g., less than 10 μm) with larger particles of the carrier (e.g., about 15 to 100 μm). Upon dispersion, the smaller particles are then respired into the lungs while the larger particles are generally retained in the mouth. Carriers suitable for blending include crystalline or amorphous excipients that have an acceptable taste and are toxicologically innocuous, whether inhaled or taken orally, e.g., the saccharides, disaccharides, and polysaccharides. Representative examples include lactose, mannitol, sucrose, xylitol and the like.

For oral administration, the pharmaceutical powders may be formulated, for example, as tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose, or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates, or sorbic acid). The preparations may also contain buffers, salts, flavorings, colorings, and sweetening agents as appropriate.

The compositions may be formulated for parenteral administration by injection, e.g., by bolus injection or continuous infusion. The active ingredient can be provided in powder form for constitution with a suitable vehicle, e.g., sterile pyrogen-free water, before use. Formulations for injection may be presented in unit dosage form, e.g., in ampules or in multi-dose containers, with an added preservative. The compositions may take such forms as suspensions, solutions or emulsions in oily or aqueous vehicles, and may contain agents such as suspending, stabilizing and/or dispersing agents.

### Adjuvants

Vaccines of the invention may be formulated with other immunoregulatory agents. In particular, vaccine compositions can include one or more adjuvants. Adjuvants that may be used in vaccine compositions described herein include, but are not limited to:

A. Mineral Containing Compositions

Mineral containing compositions suitable for use as adjuvants described herein include mineral salts, such as aluminum salts and calcium salts. Also included are mineral salts such as hydroxides (e.g., oxyhydroxides), phosphates (e.g., hydroxyphosphates, orthophosphates), sulfates, etc. (e.g., see chapters 8 & 9 of Vaccine Design (1995) eds. Powell & Newman. ISBN: 030644867X. Plenum), or mixtures of different mineral compounds (e.g., a mixture of a phosphate and a hydroxide adjuvant, optionally with an excess of the phosphate), with the compounds taking any suitable form (e.g., gel, crystalline, amorphous, etc.), and with adsorption to the salt(s) being preferred. The mineral containing compositions may also be formulated as a particle of metal salt (PCT Publication No. WO00/23105).

Aluminum salts may be included in compositions described herein such that the dose of Al³⁺ is between 0.2 and 1.0 mg per dose. In one embodiment, the aluminum-based adjuvant for use in the present compositions is alum (aluminum potassium sulfate (AlK(SO₄)₂)), or an alum derivative, such as that formed in situ by mixing an antigen in phosphate buffer with alum, followed by titration and precipitation with a base such as ammonium hydroxide or sodium hydroxide.

Another aluminum-based adjuvant for use in vaccine formulations of the present invention is aluminum hydroxide adjuvant (Al(OH)₃) or crystalline aluminum oxyhydroxide (AlOOH), which is an excellent adsorbant, having a surface area of approximately 500 m²/g. Alternatively, aluminum phosphate adjuvant (AlPO₄) or aluminum hydroxyphosphate, which contains phosphate groups in place of some or all of the hydroxyl groups of aluminum hydroxide adjuvant is provided. Preferred aluminum phosphate adjuvants provided herein are amorphous and soluble in acidic, basic and neutral media.

In another embodiment, the adjuvant for use with the present compositions comprises both aluminum phosphate and aluminum hydroxide. In a more particular embodiment thereof, the adjuvant has a greater amount of aluminum phosphate than aluminum hydroxide, such as a ratio of 2:1, 3:1, 4:1, 5:1, 6:1, 7:1, 8:1, 9:1 or greater than 9:1, by weight aluminum phosphate to aluminum hydroxide. More particularly, aluminum salts may be present at 0.4 to 1.0 mg per vaccine dose, or 0.4 to 0.8 mg per vaccine dose, or 0.5 to 0.7 mg per vaccine dose, or about 0.6 mg per vaccine dose.

Generally, the preferred aluminum-based adjuvant(s), or ratio of multiple aluminum-based adjuvants, such as aluminum phosphate to aluminum hydroxide is selected by optimization of electrostatic attraction between molecules such that the antigen carries an opposite charge as the adjuvant at the desired pH. For example, aluminum phosphate adjuvant (isoelectric point=4) adsorbs lysozyme, but not albumin at pH 7.4. Should albumin be the target, aluminum hydroxide adjuvant would be selected (isoelectric point=11.4). Alternatively, pretreatment of aluminum hydroxide with phosphate lowers its isoelectric point, making it a preferred adjuvant for more basic antigens.

B. Oil Emulsions

Oil emulsion compositions suitable for use as adjuvants in the compositions include squalene-water emulsions. Particularly preferred adjuvants are submicron oil-in-water emulsions. Preferred submicron oil-in-water emulsions for use herein are squalene/water emulsions optionally containing varying amounts of MTP-PE, such as a submicron oil-in-water emulsion containing 4-5% w/v squalene, 0.25-1.0% w/v Tween™ 80 (polyoxyelthylenesorbitan monooleate), and/or 0.25-1.0% Span™ 85 (sorbitan trioleate), and, optionally, N-acetylmuramyl-L-alanyl-D-isogluatminyl-L-alanine-2-(1′-2′-dipalmitoyl-s-n-glycero-3-huydroxyphosphosphoryloxy)-ethylamine (MTP-PE), for example, the submicron oil-in-water emulsion known as “MF59” (International Publication No. WO90/14837; U.S. Pat. Nos. 6,299,884 and 6,451,325, and Ott et al., “MF59—Design and Evaluation of a Safe and Potent Adjuvant for Human Vaccines” in Vaccine Design: The Subunit and Adjuvant Approach (Powell, M. F. and Newman, M. J. eds.) Plenum Press, New York, 1995, pp. 277-296). MF59 contains 4-5% w/v Squalene (e.g. 4.3%), 0.25-0.5% w/v Tween™ 80, and 0.5% w/v Span™ 85 and optionally contains various amounts of MTP-PE, formulated into submicron particles using a microfluidizer such as Model 110Y microfluidizer (Microfluidics, Newton, Mass.). For example, MTP-PE may be present in an amount of about 0-500 .μg/dose, more preferably 0-250 .μg/dose and most preferably, 0-100 μg/dose. For instance, “MF59-100” contains 100 μg MTP-PE per dose, and so on. MF69, another submicron oil-in-water emulsion for use herein, contains 4.3% w/v squalene, 0.25% w/v Tween™ 80, and 0.75% w/v Span™ 85 and optionally MTP-PE. Yet another submicron oil-in-water emulsion is MF75, also known as SAF, containing 10% squalene, 0.4% Tween™ 80, 5% Pluronic™-blocked polymer L121, and thr-MDP, also microfluidized into a submicron emulsion. MF75-MTP denotes an MF75 formulation that includes MTP, such as from 100-400 μg MTP-PE per dose.

Submicron oil-in-water emulsions, methods of making the same and immunostimulating agents, such as muramyl peptides, for use in the compositions, are described in detail in International Publication No. WO90/14837 and U.S. Pat. Nos. 6,299,884 and 6,451,325.

Complete Freund's adjuvant (CFA) and incomplete Freund's adjuvant (IFA) may also be used as adjuvants in the subject compositions.

C. Saponin Formulations

Saponin formulations, may also be used as adjuvants in the compositions. Saponins are a heterologous group of sterol glycosides and triterpenoid glycosides that are found in the bark, leaves, stems, roots and even flowers of a wide range of plant species. Saponins isolated from the bark of the Quillaia saponaria Molina tree have been widely studied as adjuvants. Saponins can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as immunostimulating complexes (ISCOMs).

Saponin compositions have been purified using High Performance Thin Layer Chromatography (HP-TLC) and Reversed Phase High Performance Liquid Chromatography (RP-HPLC). Specific purified fractions using these techniques have been identified, including QS7, QS17, QS18, QS21, QH-A, QH-B and QH-C. Typically, the saponin is QS21. A method of production of QS21 is disclosed in U.S. Pat. No. 5,057,540. Saponin formulations may also comprise a sterol, such as cholesterol (see, PCT Publication No. WO96/33739).

Combinations of saponins and cholesterols can be used to form unique particles called Immunostimulating Complexes (ISCOMs). ISCOMs typically also include a phospholipid such as phosphatidylethanolamine or phosphatidylcholine. Any known saponin can be used in ISCOMs. Preferably, the ISCOM includes one or more of Quil A, QHA and QHC. ISCOMs are further described in EP0109942, WO96/11711 and WO96/33739. Optionally, the ISCOMS may be devoid of (an) additional detergent(s). See WO00/07621.

A review of the development of saponin-based adjuvants can be found in Barr, et al., Advanced Drug Delivery Reviews (1998) 32:247-271. See also Sjolander, et al., Advanced Drug Delivery Reviews (1998) 32:321-338.

D. Virosomes and Virus Like Particles (VLPs)

Virosomes and Virus Like Particles (VLPs) can also be used as adjuvants with the present compositions. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Qβ-phage (such as coat proteins), GA-phage, fr-phage, AP205 phage, and Ty (such as retrotransposon Ty protein p1). VLPs are discussed further in WO03/024480, WO03/024481, and Niikura et al., Virology (2002) 293:273-280; Lenz et al., Journal of Immunology (2001) 5246-5355; Pinto, et al., Journal of Infectious Diseases (2003) 188:327-338; and Gerber et al., Journal of Virology (2001) 75(10):4752-4760. Virosomes are discussed further in, for example, Gluck et al., Vaccine (2002) 20:B10-B16. Immunopotentiating reconstituted influenza virosomes (IRIV) are used as the subunit antigen delivery system in the intranasal trivalent INFLEXAL™ product (Mischler & Metcalfe (2002) Vaccine 20 Suppl 5:B17-23) and the INFLUVAC PLUS™ product.

E. Bacterial or Microbial Derivatives

Adjuvants suitable for use in the present compositions include bacterial or microbial derivatives such as:

(1) Non-Toxic Derivatives of Enterobacterial Lipopolysaccharide (LPS)

Such derivatives include Monophosphoryl lipid A (MPL) and 3-O-deacylated MPL (3dMPL). 3dMPL is a mixture of 3 De-O-acylated monophosphoryl lipid A with 4, 5 or 6 acylated chains. A preferred “small particle” form of 3 De-O-acylated monophosphoryl lipid A is disclosed in EP 0 689 454. Such “small particles” of 3dMPL are small enough to be sterile filtered through a 0.22 micron membrane (see EP 0 689 454). Other non-toxic LPS derivatives include monophosphoryl lipid A mimics, such as aminoalkyl glucosaminide phosphate derivatives, e.g., RC-529. See Johnson et al. (1999) Bioorg. Med. Chem. Lett., 9:2273-2278.

(2) Lipid A Derivatives

Lipid A derivatives include derivatives of lipid A from Escherichia coli such as OM-174. OM-174 is described for example in Meraldi et al., Vaccine (2003) 21:2485-2491; and Pajak, et al., Vaccine (2003) 21:836-842.

(3) Immunostimulatory Oligonucleotides

Immunostimulatory oligonucleotides suitable for use as adjuvants include nucleotide sequences containing a CpG motif (a sequence containing an unmethylated cytosine followed by guanosine and linked by a phosphate bond). Bacterial double stranded RNA or oligonucleotides containing palindromic or poly(dG) sequences have also been shown to be immunostimulatory.

The CpGs can include nucleotide modifications/analogs such as phosphorothioate modifications and can be double-stranded or single-stranded. Optionally, the guanosine may be replaced with an analog such as 2′-deoxy-7-deazaguanosine. See, Kandimalla, et al., Nucleic Acids Research (2003) 31(9): 2393-2400; WO02/26757 and WO99/62923 for examples of possible analog substitutions. The adjuvant effect of CpG oligonucleotides is further discussed in Kreg, Nature Medicine (2003) 9(7): 831-835; McCluskie, et al., FEMS Immunology and Medical Microbiology (2002) 32:179-185; WO98/40100; U.S. Pat. No. 6,207,646; U.S. Pat. No. 6,239,116 and U.S. Pat. No. 6,429,199.

The CpG sequence may be directed to TLR9, such as the motif GTCGTT or TTCGTT. See, Kandimalla, et al., Biochemical Society Transactions (2003) 31 (part 3): 654-658. The CpG sequence may be specific for inducing a Th1 immune response, such as a CpG-A ODN, or it may be more specific for inducing a B cell response, such a CpG-B ODN. CpG-A and CpG-B ODNs are discussed in Blackwell, et al., J. Immunol. (2003) 170(8):4061-4068; Krieg, TRENDS in Immunology (2002) 23(2): 64-65 and WO01/95935. Typically, the CpG is a CpG-A ODN.

Typically, the CpG oligonucleotide is constructed so that the 5′ end is accessible for receptor recognition. Optionally, two CpG oligonucleotide sequences may be attached at their 3′ ends to form “immunomers.” See, for example, Kandimalla, et al., BBRC (2003) 306:948-953; Kandimalla, et al., Biochemical Society Transactions (2003) 31(part 3):664-658; Bhagat et al., BBRC (2003) 300:853-861 and WO03/035836.

(4) ADP-Ribosylating Toxins and Detoxified Derivatives Thereof.

Bacterial ADP-ribosylating toxins and detoxified derivatives thereof may be used as adjuvants in the compositions. Typically, the protein is derived from E. coli (i.e., E. coli heat labile enterotoxin “LT), cholera (“CT”), or pertussis (“PT”). The use of detoxified ADP-ribosylating toxins as mucosal adjuvants is described in WO95/17211 and as parenteral adjuvants in WO98/42375. Preferably, the adjuvant is a detoxified LT mutant such as LT-K63, LT-R72, and LTR192G. The use of ADP-ribosylating toxins and detoxified derivatives thereof, particularly LT-K63 and LT-R72, as adjuvants can be found in the following references: Beignon, et al., Infection and Immunity (2002) 70(6):3012-3019; Pizza, et al., Vaccine (2001) 19:2534-2541; Pizza, et al., Int. J. Med. Microbiol. (2000) 290(4-5):455-461; Scharton-Kersten et al., Infection and Immunity (2000) 68(9):5306-5313; Ryan et al., Infection and Immunity (1999) 67(1,2):6270-6280; Partidos et al., Immunol. Lett. (1999) 67(3):209-216; Peppoloni et al., Vaccines (2003) 2(2):285-293; and Pine et al., J. Control Release (2002) 85(1-3):263-270. Numerical reference for amino acid substitutions is typically based on the alignments of the A and B subunits of ADP-ribosylating toxins set forth in Domenighini et al., Mol. Microbiol. (1995) 15(6):1165-1167.

F. Bioadhesives and Mucoadhesives

Bioadhesives and mucoadhesives may also be used as adjuvants in the subject compositions. Suitable bioadhesives include esterified hyaluronic acid microspheres (Singh et al. (2001) J. Cont. Rele. 70:267-276) or mucoadhesives such as cross-linked derivatives of polyacrylic acid, polyvinyl alcohol, polyvinyl pyrollidone, polysaccharides and carboxymethylcellulose. Chitosan and derivatives thereof may also be used as adjuvants in the compositions. See, e.g., WO99/27960.

G. Particles

Microparticles and nanoparticles (e.g., polymeric nanoparticles) may also be used as adjuvants in the compositions. Microparticles (typically particles of ˜100 nm to ˜150 μm in diameter, e.g., ˜200 nm to ˜30 μm in diameter or ˜500 nm to ˜10 μm in diameter) and nanoparticles (typically particles of ˜10 nm to ˜1000 nm, e.g., ˜10 nm to 100 nm in diameter, ˜20 nm to ˜500 nm in diameter, or ˜50 nm to ˜300 nm in diameter) can be formed from materials that are biodegradable and non-toxic (e.g., a poly(α-hydroxy acid), a polyhydroxybutyric acid, a polyorthoester, a polyanhydride, a polycaprolactone, etc., with poly(lactide-co-glycolide). Optionally, particles can be treated to have a negatively-charged surface (e.g., with SDS) or a positively-charged surface (e.g., with a cationic detergent, such as CTAB). Particles can be engineered for specificity, such that they deliver an increased concentration of an agent to a desired location. See, e.g., Matsumoto et al., Intl. J. Pharmaceutics, 185:93-101, 1999; Williams et al., J. Controlled Release, 91:167-172, 2003; Leroux et al., J. Controlled Release, 39:339-350, 1996; Soppimath et al., J. Controlled Release, 70:1-20, 2001; Chawla et al., Intl. J. Pharmaceutics, 249:127-138, 2002; Brannon-Peppas, Intl. J. Pharmaceutics, 116, 1-9, 1995; Bodmeier et al., Intl. J. Pharmaceutics, 43:179-186, 1988; Labhasetwar et al., Adv. Drug Delivery Reviews, 24:63-85, 1997; Pinto-Alphandary et al., Intl. J. Antimicrobial Agents, 13:155-168, 2000; Potineni et al., J. Controlled Release, 86:223-234, 2003; Kost et al., Adv. Drug Delivery Reviews, 46:125-148, 2001; and Saltzman et al., Drug Discovery, 1:177-186, 2002.

Particles, preferably nanoparticles, can be assembled into structured aggregates on the micron size scale, with a shell or matrix consisting of a mixture of lipophilic and/or hydrophilic molecules (normally pharmaceutical “excipients”). The nanoparticles can be formed in the aforementioned methods and incorporate cellular material as the body of the particle, on the surface of the particles or encapsulated within the particles. The aggregate particle shell or matrix can include pharmaceutical excipients such as lipids, amino acids, sugars, polymers and may also incorporate nucleic acid and/or peptide and/or protein and/or small molecule antigens. Combinations of antigenic material can also be employed. These aggregate particles can be formed in the following methods.

U.S. patent application Ser. No. 2004/0062718 describes a method of making porous nanoparticle aggregate particles (PNAPs) for use as vaccines. Antigen can be associated with the nanoparticles by making up the nanoparticles, being bound to the surface of the nanoparticles or encapsulated within the nanoparticles or it can be incorporated in the shell of the microparticles, which then elicits both humoral and cellular immunity. Other exemplary methods of making PNAPs are described in Johnson and Prud'homme, Austral. J. Chem., 56:1021-1024, 2003.

These particles aggregate, as described by Edwards, et al., Proc. Natl. Acad. Sci. USA, 19:12001-12005, 2002, to produce larger particles of smaller subunit particles (called Trojan particles because they maintain the unique properties of their smaller subunits while also maintaining key characteristics of larger particles). The agent may be encapsulated within the subunit particles or within the larger particles made from the smaller particle aggregates.

The particles, can be in the form of a dry powder suitable for inhalation. In a particular embodiment, the particles can have a tap density of less than about 0.4 g/cm³. Particles which have a tap density of less than about 0.4 g/cm³ are referred to herein as “aerodynamically light particles.” More preferred are particles having a tap density less than about 0.1 g/cm³. Aerodynamically light particles have a preferred size, e.g., a volume median geometric diameter (VMGD) of at least about 5 microns. In one embodiment, the VMGD is from about 5 microns to about 30 microns. In another embodiment, the particles have a VMGD ranging from about 9 microns to about 30 microns. In other embodiments, the particles have a median diameter, mass median diameter (MMD), a mass median envelope diameter (MMED) or a mass median geometric diameter (MMGD) of at least 5 microns, for example from about 5 microns to about 30 microns. Aerodynamically light particles preferably have “mass median aerodynamic diameter” (MMAD), also referred to herein as “aerodynamic diameter,” between about 1 microns and about 5 microns. In one embodiment, the MMAD is between about 1 microns and about 3 microns. In another embodiment, the MMAD is between about 3 microns and about 5 microns.

In another embodiment, the particles have an envelope mass density, also referred to herein as “mass density” of less than about 0.4 g/cm³. The envelope mass density of an isotropic particle is defined as the mass of the particle divided by the minimum sphere envelope volume within which it can be enclosed.

Tap density can be measured by using instruments known to those skilled in the art such as the Dual Platform Microprocessor Controlled Tap Density Tester (Vankel, N.C.) or a Geopyc™ instrument (Micrometrics Instrument Corp., Norcross, Ga. 30093). Tap density is a standard measure of the envelope mass density. Tap density can be determined using the method of USP Bulk Density and Tapped Density, United States Pharmacopia convention, Rockville, Md., 10th Supplement, 4950-4951, 1999. Features which can contribute to low tap density include irregular surface texture and porous structure.

The diameter of the particles, for example, their VMGD, can be measured using an electrical zone sensing instrument such as a Multisizer IIe, (Coulter Electronic, Luton, Beds, England), or a laser diffraction instrument (for example Helos, manufactured by Sympatec, Princeton, N.J.). Other instruments for measuring particle diameter are well known in the art. The diameter of particles in a sample will range depending upon factors such as particle composition and methods of synthesis. The distribution of size of particles in a sample can be selected to permit optimal deposition within targeted sites within the respiratory tract.

The particles may be fabricated with the appropriate material, surface roughness, diameter and tap density for localized delivery to selected regions of the respiratory tract such as the deep lung or upper or central airways. For example, higher density or larger particles may be used for upper airway delivery, or a mixture of varying sized particles in a sample, provided with the same or different therapeutic agent may be administered to target different regions of the lung in one administration. Particles having an aerodynamic diameter ranging from about 3 to about 5 microns are preferred for delivery to the central and upper airways. Particles having an aerodynamic diameter ranging from about 1 to about 3 microns are preferred for delivery to the deep lung.

Inertial impaction and gravitational settling of aerosols are predominant deposition mechanisms in the airways and acini of the lungs during normal breathing conditions (Edwards, J. Aerosol Sci., 26: 293-317, 1995). The importance of both deposition mechanisms increases in proportion to the mass of aerosols and not to particle (or envelope) volume. Since the site of aerosol deposition in the lungs is determined by the mass of the aerosol (at least for particles of mean aerodynamic diameter greater than approximately 1 micron), diminishing the tap density by increasing particle surface irregularities and particle porosity permits the delivery of larger particle envelope volumes into the lungs, all other physical parameters being equal.

The aerodynamic diameter can be calculated to provide for maximum deposition within the lungs, previously achieved by the use of very small particles of less than about five microns in diameter, preferably between about one and about three microns, which are then subject to phagocytosis. Selection of particles which have a larger diameter, but which are sufficiently light (hence the characterization “aerodynamically light”), results in an equivalent delivery to the lungs, but the larger size particles are not phagocytosed. Improved delivery can be obtained by using particles with a rough or uneven surface relative to those with a smooth surface.

Suitable particles can be fabricated or separated, for example by filtration or centrifugation, to provide a particle sample with a preselected size distribution. For example, greater than about 30%, 50%, 70%, or 80% of the particles in a sample can have a diameter within a selected range of at least about 5 microns. The selected range within which a certain percentage of the particles must fall may be for example, between about 5 and about 30 microns, or optimally between about 5 and about 15 microns. In one preferred embodiment, at least a portion of the particles have a diameter between about 9 and about 11 microns. Optionally, the particle sample also can be fabricated wherein at least about 90%, or optionally about 95% or about 99%, have a diameter within the selected range. The presence of the higher proportion of the aerodynamically light, larger diameter particles in the particle sample enhances the delivery of therapeutic or diagnostic agents incorporated therein to the deep lung. Large diameter particles generally mean particles having a median geometric diameter of at least about 5 microns.

The preferred particles to target antigen presenting cells (“APC”) have a minimum diameter of 400 nm, the limit for phagocytosis by APCs. The preferred particles to traffic through tissues and target cells for uptake have a minimum diameter of 10 nm. The final formulation may form a dry powder that is suitable for pulmonary delivery and stable at room temperature.

H. Liposomes

Examples of liposome formulations suitable for use as adjuvants are described in U.S. Pat. No. 6,090,406, U.S. Pat. No. 5,916,588, and EP 0 626 169.

I. Polyoxyethylene Ether and Polyoxyethylene Ester Formulations

Adjuvants suitable for use in the compositions include polyoxyethylene ethers and polyoxyethylene esters. See, e.g., WO99/52549. Such formulation can further include polyoxyethylene sorbitan ester surfactants in combination with an octoxynol (WO01/21207) as well as polyoxyethylene alkyl ethers or ester surfactants in combination with at least one additional non-ionic surfactant such as an octoxynol (WO01/21152). Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.

J. Polyphosphazene (PCPP)

PCPP formulations are described, for example, in Andrianov et al., Biomaterials (1998) 19(1-3):109-115 and Payne et al., Adv. Drug. Delivery Review (1998) 31(3):185-196.

K. Muramyl Peptides

Examples of muramyl peptides suitable for use as adjuvants include N-acetyl-muramyl-L-threonyl-D-isoglutamine (thr-MDP), N-acetyl-normuramyl-1-alanyl-d-isoglutamine (nor-MDP), and N-acetylnuramyl-1-alanyl-d-isoglutaminyl-1-alanine-2-(1′-2′-dipalmitoyl-s-n-glycero-3-hydroxyphosphoryloxy)-ethylamine MTP-PE).

L. Imidazoquinoline Compounds

Examples of imidazoquinoline compounds suitable for use as adjuvants in the compositions include Imiquimod and its analogues, described further in Stanley, Clin. Exp. Dermatol. (2002) 27(7):571-577; Jones, Curr. Opin. Investig. Drugs (2003) 4(2):214-218; and U.S. Pat. Nos. 4,689,338, 5,389,640, 5,268,376, 4,929,624, 5,266,575, 5,352,784, 5,494,916, 5,482,936, 5,346,905, 5,395,937, 5,238,944, and 5,525,612.

M. Thiosemicarbazone Compounds

Examples of thiosemicarbazone compounds, as well as methods of formulating, manufacturing, and screening for compounds all suitable for use as adjuvants in the compositions include those described in WO04/60308. The thiosemicarbazones are particularly effective in the stimulation of human peripheral blood mononuclear cells for the production of cytokines, such as TNF-α.

N. Tryptanthrin Compounds

Examples of tryptanthrin compounds, as well as methods of formulating, manufacturing, and screening for compounds all suitable for use as adjuvants in the compositions include those described in WO04/64759. The tryptanthrin compounds are particularly effective in the stimulation of human peripheral blood mononuclear cells for the production of cytokines, such as TNF-α.

O. Human Immunomodulators

Human immunomodulators suitable for use as adjuvants in the compositions include cytokines, such as interleukins (e.g., IL-1, IL-2, IL-4, IL-5, IL-6, IL-7, IL-12, etc.), interferons (e.g. interferon-γ), macrophage colony stimulating factor, and tumor necrosis factor.

The compositions may also comprise combinations of aspects of one or more of the adjuvants identified above. For example, the following adjuvant compositions may be used in the invention:

(1) a saponin and an oil-in-water emulsion (WO99/11241);

(2) a saponin (e.g., QS21)+a non-toxic LPS derivative (e.g., 3dMPL) (see WO94/00153);

(3) a saponin (e.g., QS21)+a non-toxic LPS derivative (e.g., 3dMPL)+a cholesterol;

(4) a saponin (e.g., QS21)+3dMPL+IL-12 (optionally+a sterol) (WO98/57659);

(5) combinations of 3dMPL with, for example, QS21 and/or oil-in-water emulsions (See European patent applications 0835318, 0735898 and 0761231);

(6) SAF, containing 10% Squalane, 0.4% Tween™ 80, 5% Pluronic™-block polymer L121, and thr-MDP, either microfluidized into a submicron emulsion or vortexed to generate a larger particle size emulsion;

(7) Ribi™ adjuvant system (RAS), (Ribi Ininunochem) containing 2% Squalene, 0.2% Tween™ 80, and one or more bacterial cell wall components from the group consisting of monophosphorylipid A (MPL), trehalose dimycolate (TDM), and cell wall skeleton (CWS), preferably MPL+CWS (Detox™);

(8) one or more mineral salts (such as an aluminum salt)+a non-toxic derivative of LPS (such as 3dPML); and

(9) one or more mineral salts (such as an aluminum salt)+an immunostimulatory oligonucleotide (such as a nucleotide sequence including a CpG motif).

Aluminum salts and MF59 are typical adjuvants for use with injectable vaccines. Bacterial toxins and bioadhesives are typical adjuvants for use with mucosally-delivered vaccines, such as nasal or inhaled vaccines. Additional adjuvants useful in mucosal vaccines are discussed, e.g., in Stevceva and Ferrari, Curr. Pharm. Des., 11:801-11, 2005, and Cox et al., Vet. Res., 37:511-39, 2006.

### EXAMPLES

### Example 1

### Spray Drying a Suspension of M. smegmatis

To illustrate that spray drying of cellular forms without excipient leads to a powder that is too wet to produce or process, Mycobacterium smegmatis was used as a model microorganism. Dry powders were formed by spray drying using a Büchi® Mini Spray Dryer B-290 (Brinkmaim Instruments, Westbury, N.Y.) with inlet temperature, flow rate, and excipient concentration all controlled.

The microorganism was spray dried with no excipient present. A solution of pure M. smegmatis was washed in PBS-Tween® 80 and resuspended in 90 mL of water for a bacterium concentration of 3×10⁸ CFU/mL. With environmental conditions of 19.5° C. and 48% humidity, the M. smegmatis solution was spray dried with an inlet temperature of 130° C., an outlet temperature of 50° C., and a flow rate of 22 mL/min. The bacterium clump aggregated within the spray dryer cylinder and failed to emit from the cyclone as a powder. Material collected within the spray dryer was wet and nearly impossible to process.

### Example 2

### Spray Drying M. smegmatis with Leucine

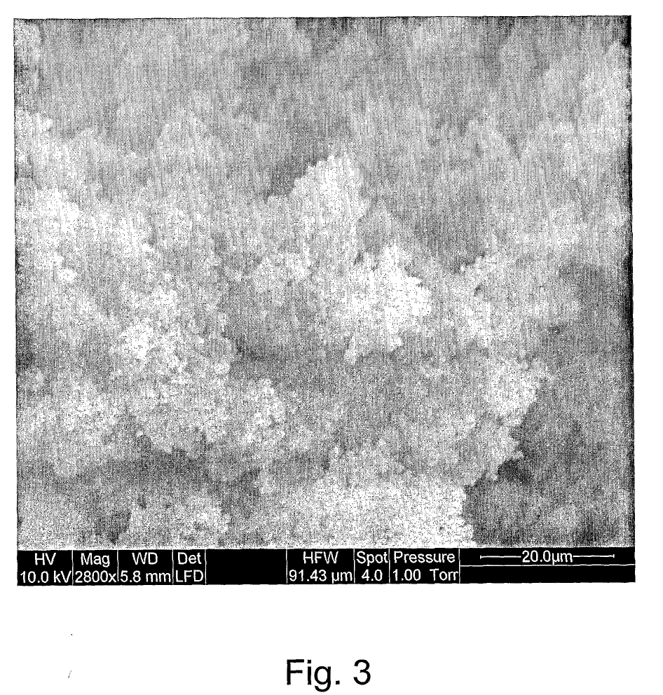

To illustrate that relatively small amounts of excipient do not lead to a successfully dried powder, M. smegmatis was spray dried using leucine as a model excipient. The dried solution consisted of 80% (by weight) of a solution of leucine at 4 mg/mL and 20% of a suspension of M. smegmatis at 3×10⁹ CFU/mL for a 400 mL solution. The solutions were mixed in-line just before reaching the spray nozzle. With environmental conditions of 20° C. and 69% humidity, the solution was spray dried with an inlet temperature of 150° C., an outlet temperature of 60° C., and a flow rate of 8 mL/min. The average droplet size was estimated at 50-60 microns. This process produced product through the cyclone of the spray dryer, but the product was excessively wet with low yield. A yellowish powder was obtained that contained viable bacteria (FIG. 3). However, this powder clumped and exhibited poor flow properties.

### Example 3

### Spray Drying M. smegmatis with Higher Concentrations of Leucine

Higher concentrations of excipient such as leucine can lead to a good spray dried powder, and even higher concentrations of excipient increase organism viability. Again, 400 ml solutions were prepared by mixing 90% and 95% of a solution of leucine at 4 mg/mL with 10% and 5% of a suspension of M. smegmatis at 3×10⁹ CFU/mL. Again, the solutions were mixed in-line just before reaching the spray nozzle. With environmental conditions of 20° C. and 69% humidity, the solutions were spray dried with an inlet temperature of 150° C., an outlet temperature of 55° C., and a flow rate of 8 mL/min. The average droplet size was estimated at 50-60 microns.

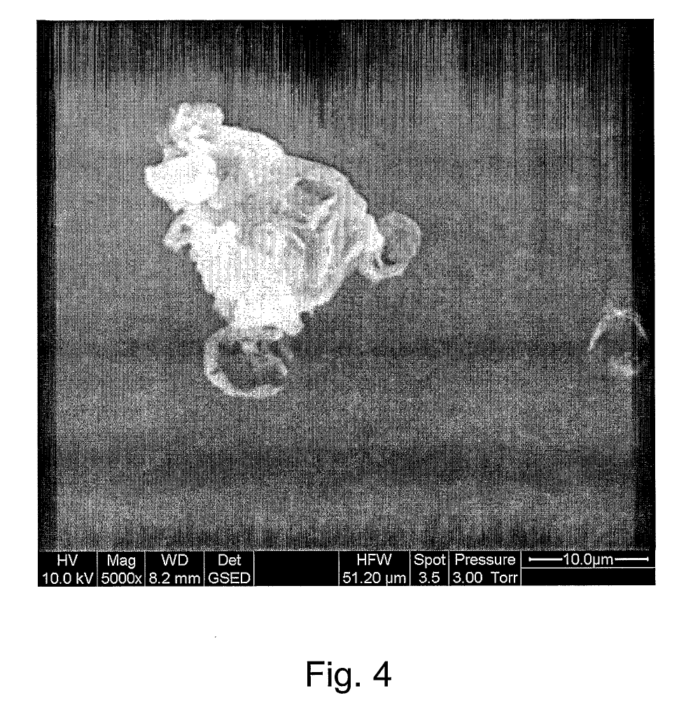

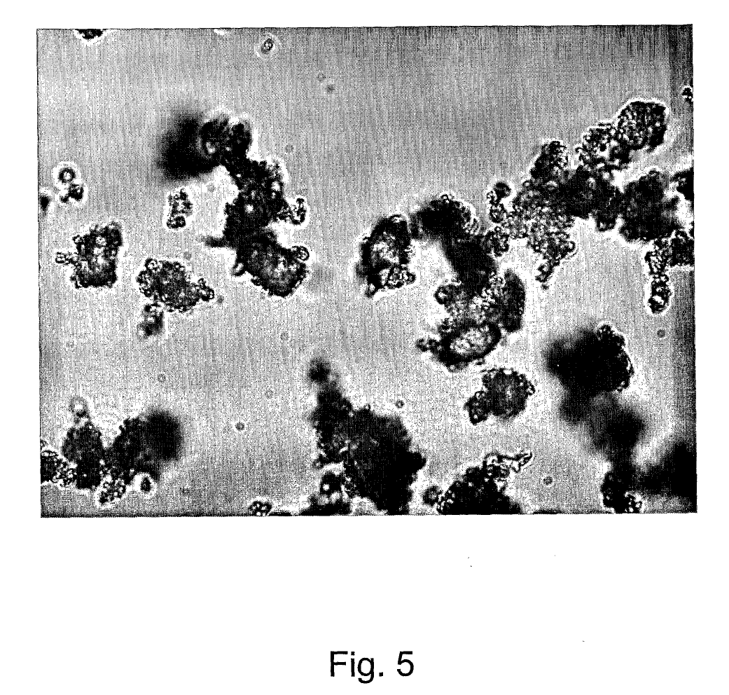

Table 1 provides results from the spray drying runs. In all cases, spray drying resulted in a fine, white viable powder, suitable for aerosol dispersion, with high product yield. Viability was measured as colony forming units on 7H9 agar plates with hygromycin. Significantly higher organism viability (about 20-80 fold) was observed for the 95:5 (leucine:smeg) powders (FIG. 4) compared to the 90:10 powders, illustrating the importance of the added excipient for protecting the microorganism during spray drying. Water content is estimated based on the gross appearance of the powder. Thermogravimetric analysis (TGA) is used for quantitative analysis of water content. FIG. 5 is a fluorescence micrograph depicting M. smegmatis that express green fluorescent protein (GFP), which were spray dried using 90:10 leucine:smeg. This micrograph shows that only a subset of the particles of the powder contain fluorescent M. smegmatis (green).

TABLE 1
Spray drying M. smegmatis with leucine

| | | | | Mass | | % | Water Content |
|---|---|---|---|---|---|---|---|
| L:Smeg | | | Mass n | out | % | Product | (1- low, 2- med, |
| Ratio | CPU in | CPU out | (mg) | (mg) | Viability | Yield | 3- high) |
| 95:5 | 1.50 × 10¹⁰ | 7.00 × 10⁸ | 1016 | 562 | 8.4% | 55.3% | 1 |
| 90:10 | 3.00 × 10¹⁰ | 2.10 × 10⁷ | 1682 | 556 | 0.2% | 33.1% | 1 |
| 95:5 | 1.50 × 10¹⁰ | 7.00 × 10⁸ | 1661 | 1651 | 4.7% | 99.4% | 2 |
| 90:10 | 3.00 × 10¹⁰ | 2.25 × 10⁷ | 1682 | 903 | 0.1% | 53.7% | 2 |

Product yield in Table 1 is measured as the proportion of mass in the final product compared to the mass of the solutes in the sprayed solution. The mass of the final product includes any residual water in the powder. Typically, some portion of the mass adheres to the drying apparatus and is not recoverable.

### Example 4

### Spray Drying M. smegmatis with Mannitol

To demonstrate that spray drying of microorganisms can be performed with other excipients, further experiments were performed using the sugar mannitol. An excipient solution consisted of 95% of a solution of mannitol at 10 mg/mL and 5% of a suspension of M. smegmatis at 3×10⁹ CFU/mL in a 200 mL solution was produced by mixing in-line just before reaching the spray nozzle. With environmental conditions of 21.9° C. and 63% humidity, the solution was spray dried with an inlet temperature of 145° C., an outlet temperature of 55° C., and a flow rate of 12 mL/min. The average droplet size was estimated at 50-60 microns. Spray drying yielded a fine, white viable powder, suitable for aerosol dispersion, with 50% product yield, which included viable bacteria.

### Example 5

### Viability of Dried M. smegmatis During Storage

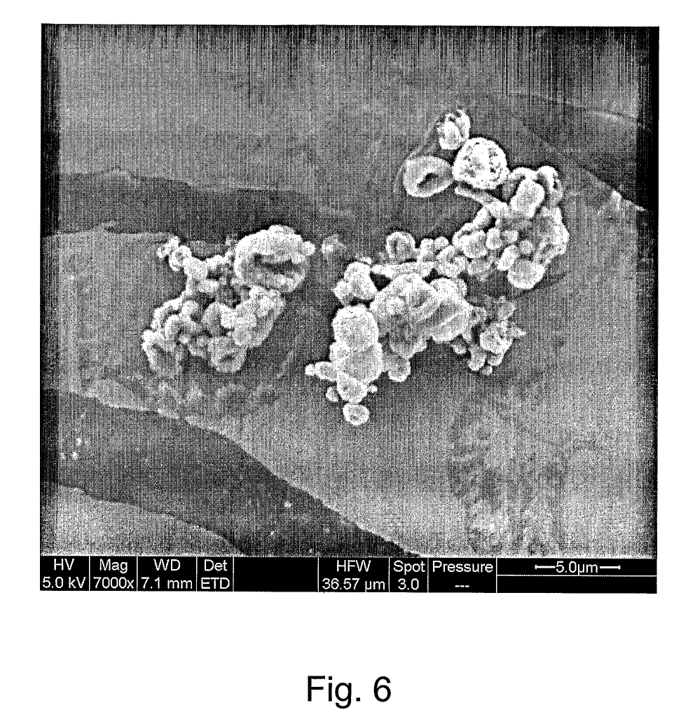

To determine the viability of spray dried M. smegmatis during storage, spray drying was performed as in Example 3, and the resulting powders were stored in sealed containers for one to two weeks at 4° C., 25° C., and 40° C. Viability was measured as colony forming units on plates. The 95:5 leucine:smeg powder retained substantial viability after one week of storage at 4° C. or 25° C., but was not significantly viable after storage at 40° C. The 90:10 leucine:smeg powder retained viability after one week of storage at 4° C., but was not viable at higher temperatures. An electron micrograph of 95:5 leucine:smeg powder after one week of storage at 25° C. is shown in FIG. 6.

### Example 6

### Modeling Spray Drying with Cryoprotectant

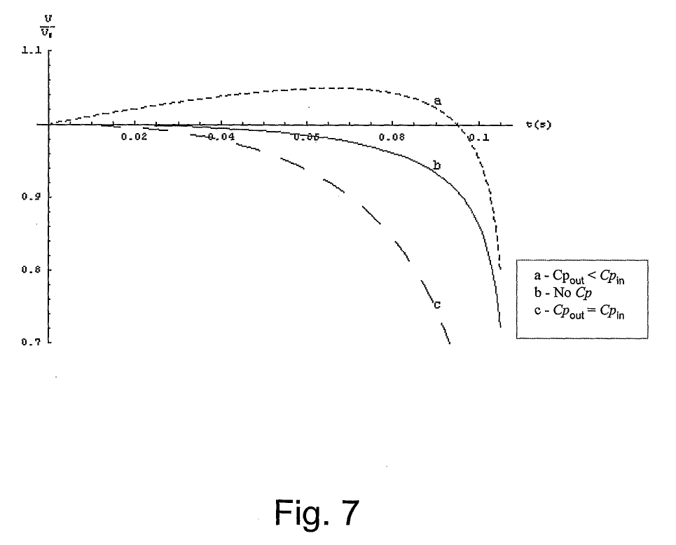

To show that the manner in which excipient is introduced during spray drying can play an important factor in retaining viability, Equation 36 was used to model the volume of a cellular material during spray drying under three different conditions: with no cryoprotectant, with equal concentrations of cryoprotectant inside and outside the cell, and with a greater concentration of cryoprotectant inside than outside the cell (FIG. 7). The objective was to show a paradigm by which membrane stress might be minimized through introduction of cryoprotectant (excipient) either within the cell, outside of the cell, or on both sides of the cell.

The modeling was done using the Mathematica® program (Wolfram, Inc., Champaign, Ill.). For all three plots, the initial cell radius (R^{c}(0)) was set at 1 μm, the initial droplet radius (R^{d}₀) was set at 25 μm, and relative cell volumes were plotted over time. Lₚ was set at 1.0 μm/(atm min); R_{gas} was set at 0.08205745867258821 (atm L)/(K mol); T was set at 295.15 K. In all three cases, k=−(K_{d}LMTD)/(λρ₁) (Eq, 33). LMTD was determined by setting an inlet temperature of 500° C., an outlet temperature of 200° C., an initial droplet temperature of 20° C. and a final droplet temperature of 65° C. These values were input to Equation 30 to give LMTD=((500° C.−20° C.)−(200° C.−65° C.))/(2.303*log₁₀((500° C.−20° C.)/(200° C.−65° C.))). K_{d} was set at 0.02 kcal/(m hr ° C.); λ was set at 530 kcal/kg; ρ₁ was set at 1000 kg/m³. The number of cells (n_{cells}) was set at 100, and the excluded volume (V_{excluded}) was set at 0.46 times the initial volume. D̅*̅_̅{̅c̅p̅}̅ was set at 10⁻⁶.

For trace (a) in FIG. 7, where the concentration of cryoprotectant is lower outside than inside the cell, the amount of extracellular salt (X^{e}ₛ) was set at 0.26 M times the initial droplet volume (V^{d}₀=4/3π(R^{d}₀)³), the amount in intracellular salt (xⁱₛ) was set at 0.26 M times the initial droplet volume, the amount of extracellular cryoprotectant (X^{e}_{cp}) was set at 0 mol, and the concentration of intracellular cryoprotectant (Cⁱ_{cp}(0)) was set at 1 M. Equation 36 was evaluated for times 0 to 0.105 seconds using these conditions to give trace (a).

For trace (b) in FIG. 7, where there is no cryoprotectant outside or inside the cell, the amount of extracellular and intracellular salt (x^{e}ₛ and xⁱₛ) were each set at 0.26 M times the initial droplet volume. The amount (X^{e}_{cp}) and concentration (Cⁱ_{cp}(0)) of intracellular cryoprotectant were set at 0 mol and 0 M, respectively. Equation 36 was evaluated for times 0 to 0.105 seconds using these conditions to give trace (b).

For trace (c) in FIG. 7, where the concentration of cryoprotectant inside the cell is equal to the concentration of cryoprotectant outside the cell, the amount of extracellular and intracellular salt (X^{e}ₛ and xⁱₛ) were set at 0.26 M times the initial droplet volume. The concentrations of cryoprotectant inside (Cⁱ_{cp}(0)) and outside the cell were set at 1 M, giving an amount of cryoprotectant outside the cell (x^{e}_{cp}) of 1 M times the initial droplet volume. Equation 36 was evaluated for times 0 to 0.105 seconds using these conditions to give trace (c).

These results show that a very different volume excursion (or membrane stress) profile is obtained depending on the method of introducing the cryoprotectant excipient. This insight can lead to methods for spray drying cellular forms that minimizes loss of cellular activity.

### Example 7

### Optimizing Cell Viability by Minimizing Membrane Osmotic Stress with M. smegmatis

To illustrate how minimization of membrane stress can improve dried cellular viability, 400 ml solutions were prepared as in Example 3 by mixing 95% of a solution of leucine at 4 mg/mL with 5% of a suspension of M. smegmatis at 3×10⁹ CFU/mL. In this case, however, glycerol was not added to the suspension M. smegmatis. These same solutions were also spray-dried without glycerol and using isotonic saline (0.9% NaCl) in place of the distilled water used in all the preceding examples. Again, the solutions were mixed in-line just before reaching the spray nozzle. With environmental conditions of 20° C. and 69% humidity, the solutions were spray dried with an inlet temperature of 150° C., an outlet temperature of 55° C., and a flow rate of 8 mL/min. The average droplet size was estimated at 50-60 microns.

TABLE 2
Spray drying 95:5 (M smegmatis/leucine)
with and without glycerol

| | | | Mass | Mass | | % |
|---|---|---|---|---|---|---|
| | | | In | out | % | Product |
| Glycerol | CPU in | CPU out | (mg) | (mg) | Viability | Yield |
| Yes | 1.50 × 10¹⁰ | 7.00 × 10⁸ | 1016 | 562 | 8.4% | 55.3% |
| No | 1.50 × 10¹⁰ | 1.93 × 10⁹ | 1520 | 830 | 24.1% | 53.5% |

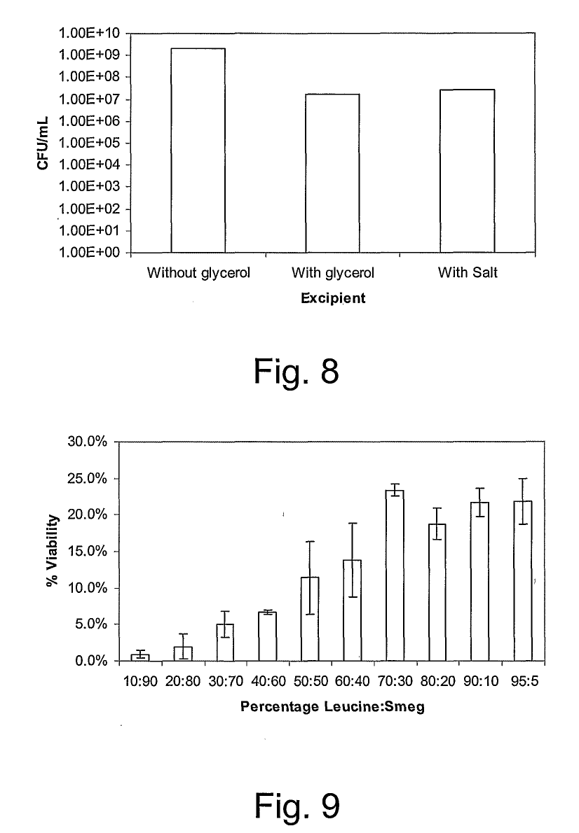

Table 2 provides results from the spray drying runs for the 95:5 leucine/smeg mixtures with and without glycerol. In all cases, spray drying resulted in a fine, white viable powder, suitable for aerosol dispersion, with high product yield. Viability was measured as colony forming units on 7H9 agar plates with hygromycin. Significantly higher organism viability was observed for the 95:5 (leucine:smeg) powders without glycerol than those with glycerol. When 95:5 (leucine:smeg) mixture was spray-dried without glycerol and with 0.9% isotonic saline, low cell viability was observed relative to the 95:5 (leucine:smeg) without glycerol and without salt (FIG. 8), illustrating the importance of removing osmotically active substances from the spray dried solution for protecting the microorganism during spray drying.

These results confirm the prediction of Example 6 that the presence of cryoprotectant or salt during the drying of a suspension of cellular material can lead to significant stress on the cellular membranes, resulting in lowered viability, presumably from cell death during spray drying.

### Example 8

### Increased Cell Content in Spray Dried Powders with High Viability of M. smegmatis

To illustrate that the retention of high viability of spray dried cells can lead to lower free water in the spray dried powder and therefore higher cell content, 400 ml solutions were prepared, as in Example 7, by mixing 90%, 50%, 40%, 30%, 20%, and 10% of a solution of leucine at 4 mg/mL with 10%, 50%, 60%, 70%, 80%, and 90% of a suspension of M. smegmatis at 3×10⁹ CFU/mL—without glycerol and without salt. Again, the solutions were mixed in-line just before reaching the spray nozzle. With environmental conditions of 20° C. and 69% humidity, the solutions were spray dried with an inlet temperature of 150° C., an outlet temperature of 55° C., and a flow rate of 8 mL/min. The average droplet size was estimated at 50-60 microns.

FIG. 9 shows viability results from the spray drying runs. As in previous examples, viability fell with lower excipient concentrations, demonstrating that high levels of excipient are required for good cellular viability. However, unlike the previous examples, fine dry powders with good viability were obtained with excipient concentrations as low as 50%. This appears to indicate that lower concentrations of excipient (lower than 90%) may provide good results when cellular integrity is maintained, and/or when no additive is used that, as in the case of glycerol, remains a liquid at room temperature. Viability was measured as colony forming units on 7H9 agar plates with hygromycin and results shown with four replicates per ratio.

These results demonstrate that elimination of cryoprotectant resulted in increased cell viability at reduced excipient concentrations.

### Example 9

### Shelf-Life Stability of Spray Dried Powders with M. smegmatis

To illustrate that viability of cells can be maintained for some period of time following drying and without freezing, the powders prepared in Example 8 with 50:50 and 95:5 leucine:M. smegmatis were placed in bulk storage conditions at 4° C., 25° C., and 40° C., and viability was measured as colony forming units on 7H9 agar plates with hygromycin.

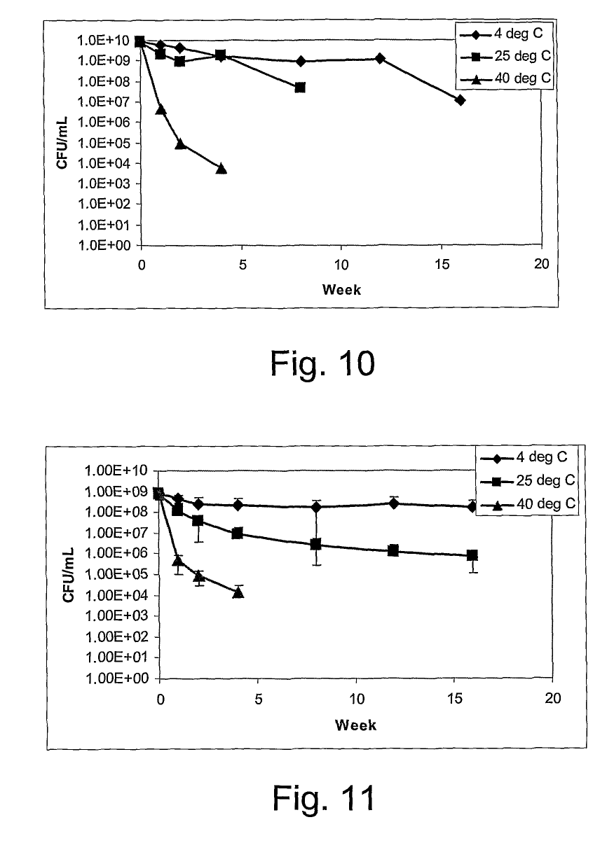

FIGS. 10 and 11 show viability results for the two powders as a function of time. Viability was maintained for several months, with the most dramatic losses in viability in the first 3 months and stabilized viability over longer time periods. Powders stored at 4° C. conditions maintained greater than a tenth of the original viability over 3 months. Powders stored at 25° C. conditions maintained viability above the 106 threshold optimal for delivery, and powders stored at 40° C. conditions maintained viability for 2 months. The difference in viability over time between the 50:50 and 95:5 powders was likely due to the difference in bacteria concentrations, which influence water content, within the powders.

### Example 10

### Effect of Stability using Monophospholipid A

The effect of a lipophilic substance, Monophospholipid A (MpLA), on stability of spray-dried M. smegmatis was determined. The experiments were conducted to find if an oily coat could be used as a method of retaining the internal water within the bacteria to increase its viability at longer time points. M. smegmatis were spray-dried as above with 95% 4 g/ml leucine solution and 5% M. smegmatis suspension, along with 0.25% MpLA. The solution was spray-dried with an inlet temp of 124° C. and an outlet temp of 45° C. Ambient conditions were 31.6° C. with 34% relative humidity. These conditions obtained a mass yield of 66%.

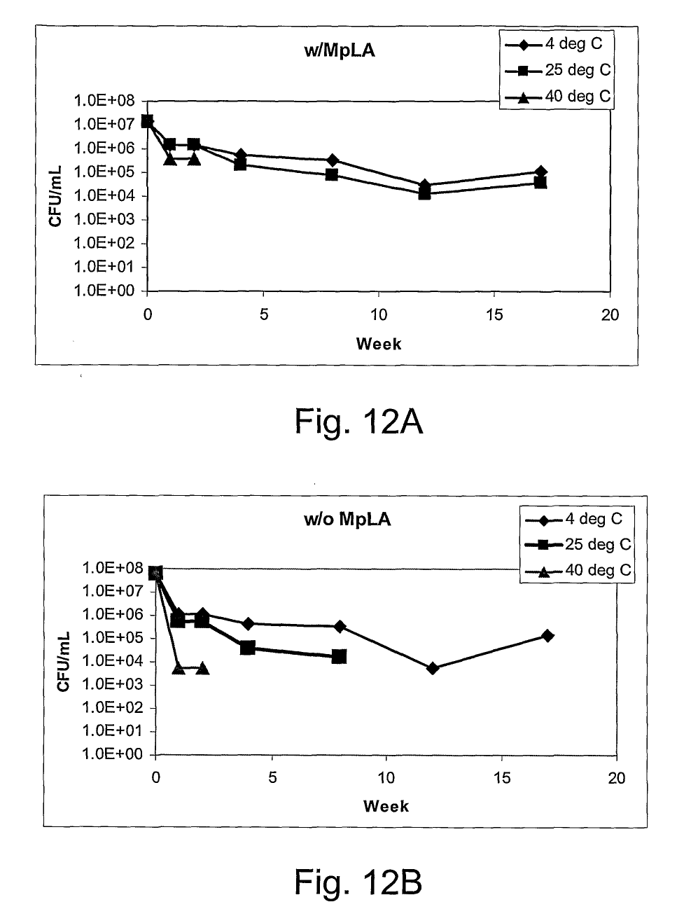

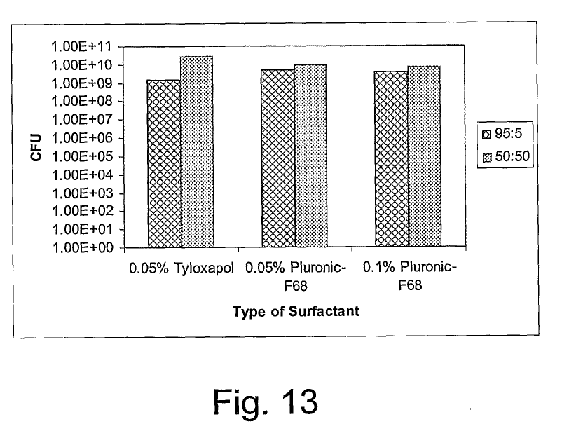

As shown in FIGS. 12A and 12B, the bacteria treated with MpLA were comparatively able to maintain viability to the non-MpLA treated bacteria over a time period of 16 weeks. Viability is measured following storage up to one year.

### Example 11

### Effect of Various Surfactants

To illustrate that the preceding results can be obtained with multiple dispersing agents without an effect on viability, the 95:5 and 50:50 smegmatis formulations were prepared using 0.05% tyloxapol (dispersing agent used in preceding examples) with 0.05% and 0.1% Pluronic™-F68. The results of these experiments are shown in FIG. 13. The use of these Pluronic™-F68 did not significantly influence the viability of the resulting powders compared to those produced using tyloxapol.

### Example 12

### Shelf-Life Stability of Spray Dried Powders with M. bovis BCG

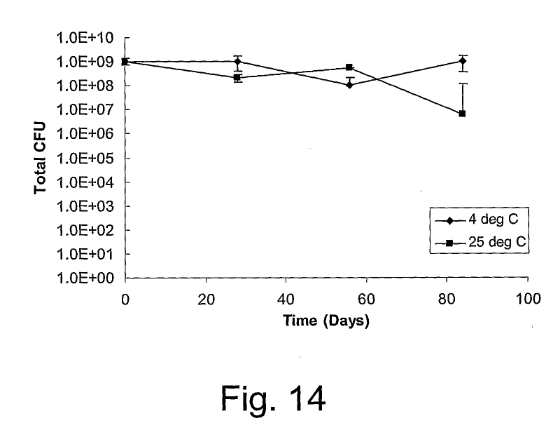

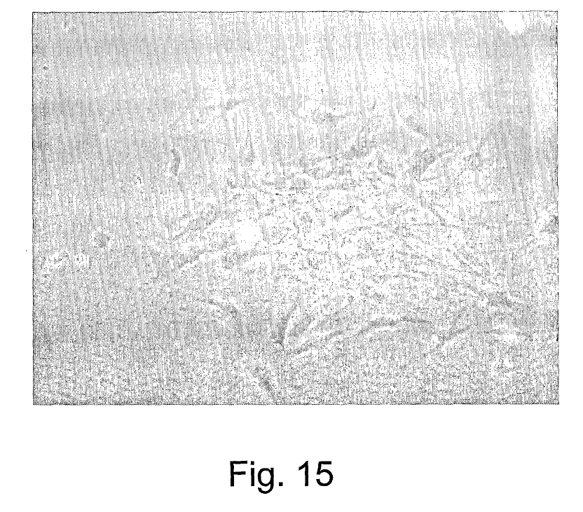

To illustrate the applicability of our conclusions to a vaccine organism, we performed similar experiments with M. bovis BCG. We prepared powders of 95:5 leucine:M. bovis BCG using the same procedure as Example 3, without salt or cryoprotectant, and placed the dried material in bulk storage conditions at 4° C., 25° C., and 40° C., and viability was measured as colony forming units on 7H9 agar plates. FIG. 14 shows viability results for the two powders as a function of time up to three months. Powders stored at 4° C. conditions largely maintained their original viability over the three months in storage. Powders stored at 25° C. conditions maintained similar viability with some loss at three months. These viability results are similar to results shown for the bacterium M. smegmatis in FIGS. 9 and 10.

### Example 13

### Spray Drying Mammalian Cells

To show that the high leucine concentration formulation with minimal membrane osmotic stress can furthermore be applied to non-bacterial cells, we have performed experiments with cultured NIH 3T3 embryonic mouse fibroblasts and primary harvest rat cardiac fibroblasts.

We prepared three formulations: we suspended 1 million fibroblast cells per milliliter with 4 milligrams of leucine per milliliter of distilled water in leucine solution/cell solution volume/volume ratios of 30/70, 50/50, and 70/30. We spray dried these formulations with conditions similar to those used in Example 3 with M. smegmatis.

All experiments indicate that primary harvest rat cardiac fibroblasts and NIH 3T3 embryonic mouse fibroblasts are roughly equal in their ability to survive the spray drying process. The higher concentration of leucine appeared to lead to greater viability on spray drying; however, given that the fibroblast cell membranes are less rigid than the bacterial membranes and more sensitive to the osmotic stress produced by intracellular osmolytically active substances, greater viability, and less net osmotic stress was obtained by spray drying cells in PBS (Table 3) or “Tyrode” solution (Table 4). Cells and leucine were both suspended in PBS or Tyrode and spray dried as above at leucine solution/cell solution volume/volume ratios of 30/70, 50/50, and 70/30. In the latter case, viable NIH 3T3 embryonic mouse fibroblasts were recovered after spray drying and observed 1 month post spray drying as shown in FIG. 15.

TABLE 3
Phosphate buffered saline (PBS) formulation

| Component | Concentration (mg/L) |
|---|---|
| Potassium phosphate monobasic | 144 |
| Sodium chloride | 9000 |
| Sodium phosphate dibasic | 795 |

TABLE 4
Tyrode's Mammalian Extracellular
Electrolyte Solution Formulation

| Component | Concentration (mg/L) |
|---|---|
| Calcium chloride | 265 |
| D-Glucose | 901 |
| HEPES | 1192 |
| Magnesium chloride | 203 |
| Potassium chloride | 403 |
| Sodium chloride | 7889 |
| Sodium phosphate | 40 |

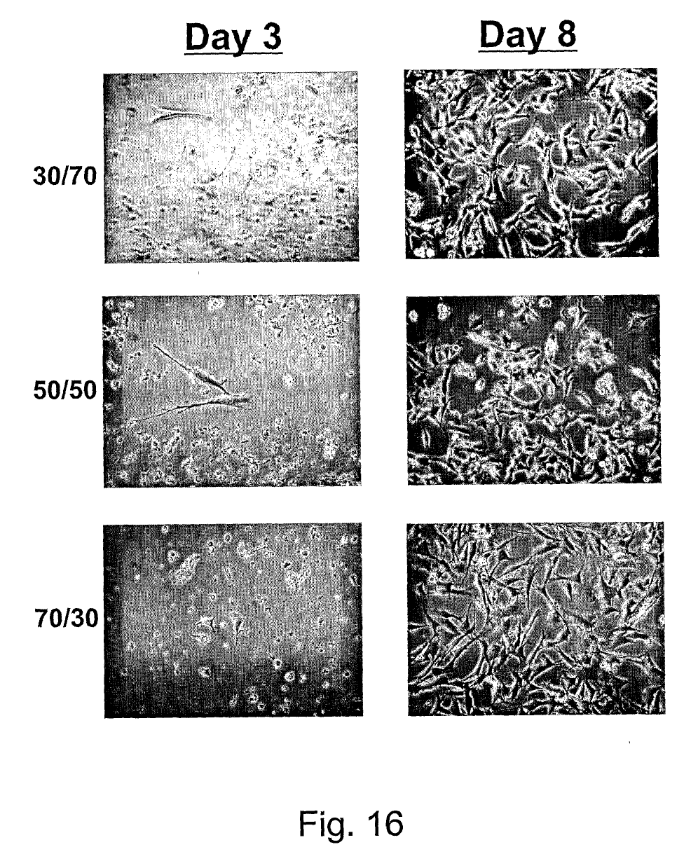

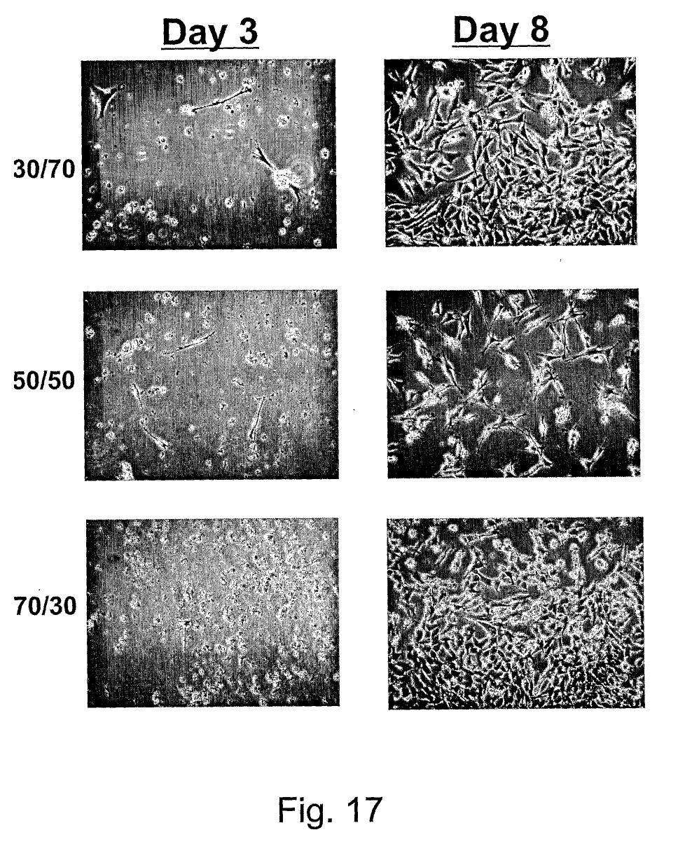

After spray drying, viable NIH 3T3 embryonic mouse fibroblasts and primary harvest rat fibroblasts were recovered from the 70/30, 50/50 and 30/70 formulations and plated. FIGS. 16 and 17 show plated cells at days 3 and 8 after spray drying. These figures show that higher excipient concentration (leucine concentration) yields higher viable cell numbers upon drying.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

1. A dry powder comprising less than about 10% water, a cellular material, and at least about 25% of an excipient by dry weight.

2. The dry powder of claim 1, wherein the powder does not comprise a significant amount of salt or cryoprotectant.

3. The dry powder of claim 1, wherein the cellular material comprises bacteria, viruses, eukaryotic microbes, mammalian cells, membrane-bound organelles, liposomes, membrane-based bioreactors, or membrane-based drug delivery systems.

4. The dry powder of claim 3 wherein the cellular material comprises bacteria.

5. The dry powder of claim 4, wherein greater than 1% of the bacteria are viable.

6. The dry powder of claim 4, wherein the bacteria are Mycobacterium tuberculosis or Mycobacterium smegmatis bacteria.

7. The dry powder of claim 4, wherein the bacteria are Bacillus Calmette-Guerin (BCG) bacteria.

8. The dry powder of claim 3, wherein the cellular material comprises mammalian cells.

9. The dry powder of claim 8, wherein the mammalian cells comprise red blood cells, stem cells, granulocytes, fibroblasts, or platelets.

10. The dry powder of claim 1, wherein the cellular material comprises living cells.

11. The dry powder of claim 1, wherein the excipient comprises leucine, mannitol, trehalose, dextran, lactose, sucrose, sorbitol, albumin, glycerol, ethanol or mixtures thereof.

12. The A method of preparing a pharmaceutical composition comprising: producing the dry powder of claim 1; and formulating the dry powder in a pharmaceutical composition.

13. The method of claim 11, wherein the pharmaceutical composition is formulated for administration by inhalation.

14. A method of producing a dry powder comprising a cellular material, the method comprising: providing an aqueous solution comprising at least 1 mg/ml excipient and at least 10⁵ units/ml of a cellular material; and spray-drying the solution under conditions to produce a dry powder with less than about 10% water by weight comprising the cellular material.

15. The method of claim 14, wherein the cellular material comprises bacteria, viruses, eukaryotic microbes, mammalian cells, membrane-bound organelles, liposomes, membrane-based bioreactors, or membrane-based drug delivery systems.

16. The method of claim 15, wherein the cellular material comprises bacteria.

17. The method of claim 16, wherein the bacteria are Mycobacterium tuberculosis or Mycobacterium smegmatis bacteria.

18. The method of claim 16, wherein the bacteria are Bacillus Calmette-Guerin (BCG) bacteria.

19. The method of claim 14, wherein the cellular material comprises mammalian cells.

20. The method of claim 19, wherein the mammalian cells comprise red blood cells, stem cells, granulocytes, fibroblasts, or platelets.

21. The method of claim 14, wherein the excipient comprises leucine, mannitol, trehalose, dextran, lactose, sucrose, sorbitol, albumin, glycerol, ethanol or mixtures thereof.

22. The method of claim 14, further comprising formulating the dry powder in a pharmaceutical composition.

23. A dry powder produced by the method of claim 14.

24. A method of spray-drying a cellular material, the method comprising:
obtaining an initial value for a radius of a unit of cellular material to be spray dried (R^{c}(0));
determining a predicted drying time;
selecting values for each of
(i) difference in inlet and outlet gas temperatures of a spray dryer (ΔT);
(ii) average droplet size (R^{d});
(iii) latent heat of vaporization of a solvent (λ);
(iv) hydraulic permeability of a membrane of the cellular material to a cryoprotectant (Lₚ);
(v) moles of extracellular solute (x^{e}ₛ);
(vi) moles of intracellular solute (xⁱₛ);
(vii) moles of extracellular cryoprotectant (x^{e}_{cp});
(viii) initial intracellular concentration of cryoprotectant (Cⁱ_{cp}(0)); and
(ix) number of cells (n_{cells});
evaluating equation 36
$$- \frac{1}{{L}_{p} {R}_{gas} T} \frac{d {R}^{c} ( t )}{d t} = \frac{{x}_{s}^{e}}{\frac{4}{3} π [ {( kt + {R}_{o}^{{d}^{2}} )}^{3 / 2} - {{n}_{cells} ( {R}^{c} ( t ) )}^{3} ]} - \frac{{x}_{s}^{i}}{\frac{4}{3} π {{R}^{c} ( t )}^{3} - {V}_{excluded}} + σ [ \frac{{x}_{cp}^{e}}{\frac{4}{3} π [ {( kt + {R}_{o}^{{d}^{2}} )}^{3 / 2} - {{n}_{cells} ( {R}^{c} ( t ) )}^{3} ]} - {C}_{cp}^{i} ( 0 ) ] 2 {\sum }_{n = 1}^{∞} \frac{{sin}^{2} ( {λ}_{n} ) - {λ}_{n} sin ( {λ}_{n} ) cos ( {λ}_{n} )}{{λ}_{n}^{2} - {λ}_{n} sin ( {λ}_{n} ) cos ( {λ}_{n} )} {e}^{- {λ}_{u}^{2} \overline{{D}_{cp}^{*}} t / {{R}^{c} ( t )}^{2}} \qquad ( 36 )$$
using the values; and
if R^{c}(t) is maintained within a minimum and maximum limit over the predicted drying time,
spray drying the cellular material using the conditions of the selected values.

25. The method of claim 24, wherein the values are selected such that damage to the cellular material during drying is minimized.

26. The method of claim 24, wherein the cellular material comprises bacteria, eukaryotic microbes, mammalian cells, membrane-bound organelles, liposomes, membrane-based bioreactors, or membrane-based drug delivery systems.

27. The method of claim 26, wherein the cellular material comprises bacteria.

28. The method of claim 27, wherein the bacteria are Mycobacterium tuberculosis or Mycobacterium smegmatis bacteria.

29. The method of claim 27, wherein the bacteria are Bacillus Calmette-Guerin (BCG) bacteria.

30. The method of claim 26, wherein the cellular material comprises mammalian cells.

31. The method of claim 30, wherein the mammalian cells comprise red blood cells, stem cells, granulocytes, or platelets.

32. The method of claim 24, wherein the method further comprises adding the cryoprotectant to the cells immediately prior to spray drying the cellular material.

33. The method of claim 32, wherein the cryoprotectant is added inside the cells.

34. The method of claim 32, wherein the cryoprotectant is added outside the cells.

35. A dry powder produced by the method of claim 25.

36. The method of claim 25, further comprising formulating the spray dried cellular material in a pharmaceutical composition.

37. A method of producing a dry powder comprising less than about 10% water by weight and bacteria of the genus Mycobacterium, the method comprising: providing an aqueous solution comprising at least 1 mg/ml excipient and at least 10⁵ colony forming units/ml of bacteria of the genus Mycobacterium; and spray-drying the solution under conditions to produce a dry powder comprising less than about 10% water by weight and bacteria of the genus Mycobacterium.

38. The method of claim 37, wherein the aqueous solution does not contain added salt or cryoprotectant.

39. A dry powder produced by the method of claim 37.

40. The method of claim 37, further comprising formulating the dry powder in a pharmaceutical composition.
